(19) United States
(12) Patent Application Publication
(10) Pub. No.: US 2026/0077006 A1
(43) Pub. Date: Mar. 19, 2026

(54) ONCOLYTIC VIRUS EXPRESSING AN IMMUNE CELL ENGAGER FOR TUMOR TARGETING

(71) Applicant: VIROGIN BIOTECH CANADA LTD, Richmond (CA)

(72) Inventors: Xiaohu Liu, Vancouver (CA); I-Fang Lee, Vancouver (CA); Yanal M. Murad, Vancouver (CA)

(21) Appl. No.: 19/110,072

(22) PCT Filed: Sep. 8, 2023
(86) PCT No.: PCT/US2023/073810

§ 371 Date: Mar. 8, 2025

Related U.S. Application Data
(60) Provisional application No. 63/375,033, filed on Sep. 8, 2022.

(51) Int. Cl. A61K 35/763; A61P 35/00; C07K 14/005; C07K 16/28; C07K 16/30; C12N 7/00; C12N 15/113

(52) U.S. Cl. CPC A61K 35/763; A61P 35/00; C07K 14/005; C07K 16/2803; C07K 16/2809; C07K 16/2818; C07K 16/2827; C07K 16/2875; C07K 16/2878; C07K 16/3092; C12N 7/00; C12N 15/1133; C07K 2317/31; C07K 2317/569; C07K 2317/622; C12N 2310/141; C12N 2710/16621; C12N 2710/16622; C12N 2710/16632

(57) ABSTRACT

An HSV vector with an expression cassette encoding an immune cell engager protein under control of an EF1α promoter. ICP27 under control of a CEA promoter. ICP34.5 under control of miRNA-124/143, and ICP47 under control of a ICP27 promoter is disclosed. The HSV vector also includes deletion of one copy of terminal repeat long region and one copy of terminal repeat short region. The immune cell engager protein comprises a T-cell binding domain and a tumor-associated antigen binding domain.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD OF THE INVENTION

The present invention relates generally to oncolytic viral vectors that express molecules that target tumor cells.

### REFERENCE TO SEQUENCE LISTING, TABLE OR COMPUTER PROGRAM

The contents of the electronic sequence listing (VIRO_420P1_sequencelisting.text.xml; Size: 14.4 bytes and Date of Creation: Jul. 18, 2022) is herein incorporated by reference in its entirety.

### BACKGROUND

Redirecting the activity of T cells to target tumor cells is a powerful tool for cancer immunotherapy. Bispecific T-cell engagers (BiTEs) can potentiate T cell activation by binding to a selected antigen on tumor cells and directly bridging said tumor cells to the CD3 epsilon chain located within the T cell receptor (TCR) complex on T cells, thus bypassing both the major histocompatibility complex (MHC) and intrinsic TCR specificity. Systemic delivery of BiTEs has been linked to neurotoxicity and cytokine release syndrome. Thus there is a need in the art for improved therapeutic compositions and methods that ameliorate BiTE-mediated toxicity during cancer therapy.

The present invention overcomes shortcomings of current cancer therapies, including immunotherapies, and further provides additional unexpected benefits.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention relates to compositions and methods for treating cancer with recombinant oncolytic viral vectors. Within one embodiment of the invention recombinant herpes simplex viruses are provided comprising a modified oncolytic herpes virus genome, wherein the modified herpes virus genome comprises an expression cassette encoding An immune cell engager protein, wherein the immune cell engager protein comprises a T-cell binding domain and a tumor-associated antigen binding domain.

Also provided are therapeutic compositions comprising the recombinant herpes viruses described herein, as well as methods of lysing tumor cells, and methods of treating cancers in a subject comprising the step of administering one of the recombinant herpes viruses described herein to a subject.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described examples with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 diagrammatically depicts the overall structural organization of the double-stranded deoxyribonucleic acid (DNA) elements of VG301.

FIGS. 2A, 2B and 2C are charts that shows alignment of various anti-CEACAM6 antibodies, including: alignment of VHH (single domain antibodies with consensus highlighting (FIG. 2A panel 1), or, physico-chemical property highlighting (FIG. 2A panel 2); scFv VH with consensus highlighting (FIG. 2B panel 1); and or, physico-chemical property highlighting (FIG. 2B panel 2); and scFv VL with consensus highlighting (FIG. 2C panel 1); and or, physico-chemical property highlighting (FIG. 2C panel 2).

FIG. 3 illustrates 14 different embodiments of using an intratumorally injected or OV-delivered secretable immune cell engager (ICE) protein to tag cells expressing Muc16 or CD47 for immune destruction. The relative arrangement of functional elements within each ICE embodiment is depicted as a series of boxes, with each box representing either a target antigen recognition domain such as the anti-Muc16 (αMuc16) antibody J97 or a fragment of SIRPa that is recognized by the regulatory protein CD47, an effector cell activation domain such as the anti-CD3 (αCD3) antibody UCHT or enhanced Fc (eFc) that is recognized by Fc-gamma receptors on effector cells, or a therapeutic domain such as the anti-PD-L1 (αPD-L1) neutralizing antibody (NAb) LH66, with each domain joined to adjacent domains using linker polypeptides such as the common linker amino acid sequence GGGGS or variants thereof. The name of each engager is comprised of the names of each functional element arranged in the order in which they appear and separated by dashes.

FIGS. 4A and 4B are bar graphs depicting cytolysis at 24 hours post-treatment in the Muc16 medium expression cell line HeLA that was co-cultured with PBMC from two different donors (PBMC 1 and PBMC 2) and treated with four different immune cell engager proteins targeting Muc16 at four different engager concentrations (no engager, 0.5 pM, 5 PM, or 50 pM).

FIGS. 4C and 4D are bar graphs depicting cytolysis at 24 hours post-treatment in the Muc16 high expression cell line OVCAR3 that was co-cultured with PBMC from two different donors (PBMC 1 and PBMC 2) and treated with four different immune cell engager proteins targeting Muc16 at four different engager concentrations (no engager, 0.5 pM, 5 pM, or 50 pM).

FIGS. 5A and 5B are bar graphs depicting cytolysis at 24 hours post-treatment in the Muc16 medium expression cell line HeLA that was co-cultured with PBMC from two different donors (PBMC 1 and PBMC 2) and treated with six different immune cell engager proteins targeting Muc16 at four different engager concentrations (no engager, 0.5 pM, 5 pM, or 50 pM).

FIGS. 5C and SD are bar graphs depicting cytolysis at 24 hours post-treatment in the Muc16 high expression cell line OVCAR3 that was co-cultured with PBMC from two different donors (PBMC 1 and PBMC 2) and treated with six different immune cell engager proteins targeting Muc16 at four different engager concentrations (no engager, 0.5 pM, 5 pM, or 50 pM).

FIGS. 6A, 6B, and 6C are bar graphs depicting cytolysis at 24 hours, 48 hours, and 72 hours post-treatment in the Muc16 high expression cell line OVCAR3, the MUC16 medium expression cell line HeLA, and the Muc16 low expression cell line PC3, respectively, that were co-cultured with PBMC from two different donors and treated with the Muc16-targeting immune cell engager protein UCHT-J97-LH66-eFc-SIRPA at two different engager concentrations (no engager or 5 pM).

FIG. 7A is a bar graph depicting the concentration of interferon-gamma (IFNg), interleukin-2 (IL-2), and granzyme B (GrzB) as measured by ELISA from Muc16 high expression OVCAR3 cells co-cultured with PBMC from two different donors and treated with the Muc16-targeting immune cell engager protein UCHT-J97-LH66-eFc-SIRPA at two different engager concentrations (no engager or 5 pM).

FIGS. 78, 7C, and 7D are bar graphs depicting the level of expression of selected lymphocyte surface activation markers on CD3+ T cells, CD56+ NK cells, and CD19+ B cells, respectively, as measured by flow cytometry from Muc16 high expression OVCAR3 cells co-cultured with PBMC from two different donors and treated with the Muc16-targeting immune cell engager protein UCHT-J97-LH66-eFc-SIRPA at two different engager concentrations (no engager or 5 pM).

FIG. 8A is a bar graph depicting cytolysis at 24 hours, 48 hours, and 72 hours post-treatment in the Muc16 high expression cell line OVCAR3 co-cultured with pan-T cells from two different donors and treated with the Muc16-targeting immune cell engager protein UCHT-J97-LH66-eFc-SIRPA at two different engager concentrations (no engager or 5 pM).

FIG. 8B is a bar graph depicting the level of expression of selected lymphocyte surface activation markers including CD69, 4-1BB, and PD-1 as measured by flow cytometry from Muc16 high expression OVCAR3 cells co-cultured with pan-T cells from two different donors and treated with the Muc16-targeting immune cell engager protein UCHT-J97-LH66-eFc-SIRPA at two different engager concentrations (no engager or 5 pM).

FIG. 8C is a bar graph depicting the concentration of interferon-gamma (IFNg), interleukin-2 (IL-2), and granzyme B (GrzB) as measured by ELISA from Muc16 high expression OVCAR3 cells co-cultured with pan-T cells from two different donors and treated with the Muc16-targeting immune cell engager protein UCHT-J97-LH66-eFc-SIRPA at two different engager concentrations (no engager or 5 pM).

FIG. 9A is a bar graph depicting antibody-dependent cellular cytotoxicity (ADCC) in the Muc16 high expression cell line OVCAR3 co-cultured with NK cells from two different donors and treated with the Muc16-targeting immune cell engager (ICE) protein UCHT-J97-LH66-eFc-SIRPA at two different engager concentrations (no engager or 1 μg/mL).

FIG. 9B is a bar graph depicting the level of expression of selected NK cell surface activation markers including CD69 and CD107 as measured by flow cytometry from Muc16 high expression OVCAR3 cells co-cultured with NK ceils from two different donors and treated with the Muc16-targeting immune cell engager (ICE) protein UCHT-J97-LH66-eFc-SIRPA at two different engager concentrations (no engager or 1 μg/mL).

FIG. 9C is a bar graph depicting the concentration of interferon-gamma (IFNg) and granzyme B (GrzB) as measured by ELISA from Muc16 high expression OVCAR3 cells co-cultured with NK cells from two different donors and treated with the Muc16-targeting immune cell engager protein UCHT-J97-LH66-eFc-SIRPA at two different engager concentrations (no engager or 1 μg/mL).

FIG. 9D is a bar graph depicting antibody-dependent cellular phagocytosis (ADCP) in the Muc16 high expression cell line OVCAR3 co-cultured with undifferentiated M0 macrophages (OVCAR3+M0) or with pro-inflammatory M1 macrophages (OVCAR3+M1) and treated with the Muc16-targeting immune cell engager (ICE) protein UCHT-J97-LH66-eFc-SIRPA at two different engager concentrations (no engager or 5 μg/mL)

FIG. 10 illustrates 7 different embodiments of using an intratumorally injected or OV-delivered secretable immune cell engager (ICE) protein to tag cells expressing programmed death-ligand 1 (PD-L1) or CD47 for immune destruction. The relative arrangement of functional elements within each ICE embodiment is depicted as a series of boxes, with each box representing either a target antigen recognition domain such as the anti-PD-L1 (αPD-L1) antibody LH56 or a fragment of SIRPa that is recognized by the regulatory protein CD47, an effector cell activation domain such as the anti-CD3 (αCD3) antibody UCHT or enhanced Fc (eFc) that is recognized by Fc-gamma receptors on effector cells, or a therapeutic domain such as the anti-PD-L1 (αPD-L1) neutralizing antibody (NAb) LH66, with each domain joined to adjacent domains using linker polypeptides such as the common linker amino acid sequence GGGGS or variants thereof. The name of each engager is comprised of the names of each functional element arranged in the order in which they appear and separated by dashes.

FIGS. 11A, 11B, and 11C are bar graphs depicting cytolysis at 24 hours post-treatment in the PD-L1 high expression cell line JIMT-1, the PD-L1 medium expression cell line A549, and the PD-L1 low expression cell line PC3, respectively, that were co-cultured with PBMC and treated with seven different PD-L1-targeting immune cell engager proteins at three different engager concentrations (no engager, 5 pM, or 50 pM).

FIG. 12A is a bar graph depicting antibody-dependent cellular cytotoxicity (ADCC) in the PD-L1 high expression cell line JIMT-1, the PD-L1 medium expression cell line A549, and the PD-L1 low expression cell line PC3 co-cultured with NK cells from two different donors and treated with the PD-L1-targeting immune cell engager (ICE) protein UCHT-LH56-LH66-eFc-SIRPA at two different engager concentrations (no engager or 40 nM).

FIG. 12B is a bar graph depicting the proportion of CD69+ NK cells as measured by flow cytometry from PD-L1 high expression JIMT-1 cells, PD-L1 medium expression A549 cells, and PD-L1 low expression PC3 cells co-cultured with NK cells from two different donors and treated with the PD-L1-targeting immune cell engager (ICE) protein UCHT-LH56-LH66-eFc-SIRPA at two different engager concentrations (no engager or 40 nM).

FIG. 13 illustrates two different embodiments of using an intratumorally injected or OV-delivered secretable immune cell engager (ICE) protein to tag cells expressing transferrin receptor 1 (TfR1), programmed death-ligand 1 (PD-L1), or CD47 for immune destruction. The relative arrangement of functional elements within each ICE embodiment is depicted as a series of boxes, with each box representing either a target antigen recognition domain such as the anti-TfR (αTfR) antibody H7 or anti-PD-L1 (αPD-L1) antibody LH56 or a fragment of SIRPa that is recognized by the regulatory protein CD47, or an effector cell activation domain such as the anti-CD3 (αCD3) antibody UCHT or enhanced Fc (eFc) that is recognized by Fc-gamma receptors on effector cells, with each domain joined to adjacent domains using linker polypeptides such as the common linker amino acid sequence GGGGS or variants thereof. The name of each engager is comprised of the names of each functional element arranged in the order in which they appear and separated by dashes.

FIG. 14 is a bar graph depicting cytolysis at 48 hours post-treatment in the TfR-expressing cell line A549 that was co-cultured with PBMC with 4 different E/T (effector:target) ratios and treated with two different TfR-targeting immune cell engager proteins at 50 pM (control group remained untreated).

FIG. 15 is a bar graph depicting cytolysis at 72 hours post-treatment in the TfR-expressing cell lines PC3, HeLA, MCF7, OVCAR3, and HCT116 that were co-cultured with PBMC from two different donors and treated with the TfR-targeting immune cell engager protein UCHT-LH56-eFc-SIRPA-H7 at two different engager concentrations (no engager or 5 pM).

### DETAILED DESCRIPTION OF THE INVENTION

The present invention is generally directed to oncolytic viruses (e.g., a HSV-1 or HSV-2 oncolytic virus or “OV”)) engineered to express and deliver an immune cell engagers molecule (ICE) to a tumor target. The virus may also be engineered to express a variety of other immunomodulators, such as IL-12, IL-15, and IL-15RA1, and it may be modified to enable tumor-specific virus replication and/or tumor-specific payload expression. The immune cell engager promotes T-cell activation in the presence of cells displaying a selected tumor-associated antigen (TAA) on the cell surface. Consequently, the OV expressing a tumor-specific BiTE would be preferentially used to treat tumors that overexpress the selected tumor-associated antigen used for the tumor-targeting function of the ICE.

The use of an oncolytic virus to express an immune cell engager combines two complementary cancer immunotherapies to synergistically enhance antitumor activity. While an OV can only infect a small number of cells in the tumor mass, it serves to prime the tumor microenvironment for immunotherapy by increasing the levels of tumor-infiltrating lymphocytes, by the lytic destruction of a subset of tumor cells to release tumor associated antigens (TAAs) that may facilitate an anti-tumor adaptive response, and by providing a danger signal that may partially counteract the immunosuppressive tumor microenvironment. BiTEs can further be utilized to enhance the potency and target range of CAR T-cell therapy by targeting tumor cells expressing a particular TAA that is different from the CAR-targeted TAA, resulting in activation of both nonspecific CAR-negative and CAR-positive T-cells within the tumor mass. The OV can also be used to deliver therapeutic payloads that can be secreted from infected cells to spread throughout the tumor and its surrounding stroma.

In order to further an understanding of the various embodiments herein, the following sections are provided which describe various embodiments: A. Immune Cell Engager; B. Oncolytic Viruses; C. Specific Herpes Virus Constructs-VG301; D. Post-transcriptional Regulation; E. Expression of ICP27 in VG301 is Transcriptionally Controlled; F. Payload Expression of VG301 is Enhanced; G. Truncated Glycoprotein B; H. Modified ICP47 Promoter; I. Alternative Compositions and Methods related to Immune Cell Engager Proteins; J. Therapeutic Compositions; and K. Administration.

a. Immune Cell Engager

Immune cell engagers (ICE) are designed to redirect and activate immune effector cells, utilizing at least one arm to target one or more tumor-associated antigens and another arm or multiple arms directed against one or more activating receptors in immune effector cells. ICE includes, for example, Bispecific T-cell engagers which are secretable fusion proteins that include a T-cell binding domain (e.g., an anti-CD3, anti-CD28, anti-OX40, or an anti-4-1BB antibody or antibody fragment) and a tumor-associated antigen binding domain (e.g., anti-TAA antibody or antibody fragment) joined by a flexible linker.

As used herein the term “antibody fragment” refers to any binding fragment of naturally occurring full-length immunoglobulins (i.e., naturally occurring or recombinantly formed whole molecules) (e.g., an IgG antibody such as IgG1, IgG2a, IgG3, IgG4 (and IgG4 subforms), IgA isotypes, IgE and IgM) or recombinantly produced antibody. Representative examples of antibody fragments or segments include separate heavy chains, light chains, and portions of an antibody such as F(ab′)₂, F(ab)₂, Fab′, Fab, Fv, scFv (single chain Fv) and the like, including the half-molecules of IgG4 (see van der Neut Kolfschoten et al. (Science 2007; 317 (14 September): 1554-1557). Antibody fragments or segments also include immunologically active minimal recognition units consisting of the amino acid residues that mimic the hypervariable region, such as CDRs. Antibody fragments or segments can be produced by enzymatic or chemical separation of intact immunoglobulins, or by recombinant techniques. The term “antibody” should also be understood to include one or more immunoglobulin chains that are chemically conjugated to or expressed as fusion proteins along with other proteins.

The term “antibody fragment” also includes single domain antibodies (sdAbs) and nanobodies. SdAbs are comprised of a single monomeric variable region and may be derived from either heavy chains or light chains. One advantage of sdAbs over traditional monoclonal antibodies or other antibodies such as scFv or diabodies is the significantly smaller size (˜2 nm) of sdAbs, thus making them less likely to interfere with the functions of essential glycoproteins on the viral envelope due to steric hindrance. In addition, sdAbs and nanobodies exhibit high affinity towards their targets and excellent biophysical properties such as thermal stability.

The T-cell binding domain of the bispecific T-cell engager molecule can include any antibody or antibody fragment designed to target, for example, the human CD3 antigen/TCR complex. Examples of suitable CD3-binding antibodies are the mouse monoclonal IgG1 antibody UCHT1 (SEQ ID NO:1), which is active against both human and chimpanzee CD3, the mouse monoclonal IgG3 antibody SP34 (SEQ ID NO:2), which is active against CD3 from humans and from both cynomolgus and rhesus monkeys, and the mouse monoclonal IgG2a antibody CRIS-7 (SEQ ID NO:3), which can also cross-react with human and rhesus monkey CD3. This list is not exhaustive and other commercially available CD3-binding antibodies can be used as part of the T cell binding domain of the BiTE as long as the CD3-binding antibodies possess the ability to trigger T cell activation.

The tumor antigen-binding domain of the immune cell engager can be an antibody or antibody fragment designed to target a broad array of antigens overexpressed in solid tumors. Representative examples include B7-H3, B7-H6, CD70, CEA, CEACAM6, CSPG4, EGFRvIII, EphA2, EpCAM, EGFR, ErbB2 (HER2), FAP, FRα, GD2, GD3, HLA-A1+MAGE1, IL-11Rα, IL-13Rα2, Lewis-Y, Mesothelin, Muc1, Muc16, NKG2D, PSMA, ROR1, TAG72, PD-L1 and VEGFR2. A more ubiquitous and broad-spectrum tumor-associated cell surface antigen, such as TfR1 or GLUT1 may also be used for the tumor antigen-binding arm of the ICE.

Within one embodiment of the invention an anti-TfR1 scFv H7 sequence is provided comprising the following consensus sequence (wherein the VH sequence is provided in underline, a linker is bolded, and the VL sequence is italicized):

QVQLQESGGGVVQPGRSLRLSCAASRFTFSSYAMHWVRQAPGKGLEWVA
VISYDGSNKYYADSVKGRFTISRDNSKNTLYLQMNSLRAEDTAVYYCAR
DLSGYGDYPDYWGQGTLVTVSSGGGGSGGGGSGGGGSSELTQDPAVSVA
LGQTVRITCQGDSLRSYYASWYQQKPGQAPVLVMYGRNERPSGVPDRFS
GSKSGTSASLAISGLQPEDEANYYCAGWDDSLTGPVFGGGTKLTVLG.

Within another embodiment of the invention an anti-HER2 scFv sequence is provided comprising the following consensus sequence (wherein the VH sequence is provided in underline, a linker is bolded, and the VL sequence is italicized):

DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKGGGGSGGGGSGGGGSGGGGSEVQLVESGGGLVQPGGSLRL
SCAASGFNIKDTYIHWVRQAPGKGLEWVARIYPTNGYTRYADSVKGRFT
ISADTSKNTAYLQMNSLRAEDTAVYYCSRWGGDGFYAMDYWGQGTLVTV
SS.

Within another embodiment of the invention an anti-EpCAM scFv 3-10 sequence is provided comprising the following consensus sequence (wherein the VH sequence is provided in underline, a linker is bolded, and the VL sequence is italicized):

EVQLLEQSGAELVKPGASVKISCKASGYAFTNYWLGWVKQRPGHGLEWI
GDLFPGSGNTHYNERFRGKATLTADKSSSTAFMQLSSLTSEDSAVYFCA
RLRNWDEAMDYWGQGTTVTVSSGGGGSGGGGSGGGGSELVMTQSPSYLA
ASPGETITINCRASKSISKYLAWYQEKPGKTNKLLIYSGSTLQSGIPSR
FSGSGSGTDFTLTISSLEPEDFAMYYCQQHNEYPYTFGGGTKLEIK.

Within another embodiment of the invention an anti-EpCAM scFv 5-10 sequence is provided comprising the following consensus sequence (wherein the VH sequence is provided in underline, a linker is bolded, and the VL sequence is italicized):

EVQLLEQSGAELVRPGTSVKISCKASGYAFTNYWLGWVKQRPGHGLEWI
GDIFPGSGNIHYNEKFKGKATLTADKSSSTAYMQLSSLTFEDSAVYFCA
RLRNWDEPMDYWGQGTTVTVSSGGGGSGGGGSGGGGSELVMTQSPSSLT
VTAGEKVTMSCKSSQSLLNSGNQKNYLTWYQQKPGQPPKLLIYWASTRE
SGVPDRFTGSGSGTDFTLTISSVQAEDLAVYYCQNDYSYPLTFGAGTKL
EIK.

Within another embodiment of the invention, an anti-PD-L1 antibody sequence (LH56) is provided comprising the following consensus sequence:

QVQLVESGGGLVQAGGSLRLSCAASPRTFNNYAMAWFRQAPGKEREFVA
RIRWSSGTTFYQESVKGRFTISGDNAENTVYLQMDSLKPEDTAVYYCAG
ATGGGNYAFTAENYYSYWGQGTQVTVSS.

Within another embodiment of the invention, an anti-PD-L1 neutralizing antibody sequence (LH66) is provided comprising the following consensus sequence:

QVKLEESGGGLVQAGGSLRLSCVASGLTFNTYFMAWFRQAPGKEREFVA
TISEDGGYIYYKDSVKGRFTISRDNARDTVYLQMNSLKPEDTAVYYCAA
DRTGSSGISTPGRYWGRGTQVTVSS.

Within another embodiment of the invention, an anti-Muc16 antibody (J97) sequence is provided comprising the following consensus sequence:

AQVQLVESGGGSVQAGGSLRLSCAASGGTFSPYRMSWFRQAPGKEREFV
ATITWGAGTTYFRDSVKGRFAISRDIANYTMYLQMNSLKPDDTAVYYCA
AANRPNLVIDSGFYDYWGQGTQVTVSS.

Within another embodiment of the invention, an enhanced Fc (eFc) sequence is provided comprising the following consensus sequence:

EPKSCDKTHTCPPCPAPELLGGPDVFLFPPKPKDTLMISRTPEVTCVVV
DVSHEDPEVKFNWYVDGVEVHNAKTKPREEQYNSTYRVVSVLTVLHQDW
LNGKEYKCKVSNKALPLPEEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEGLHNHYTQKSLSLSPGK.

Within another embodiment of the invention, a fragment of SIRPA is provided comprising the following consensus sequence:

EEELQVIQPDKSVLVAAGETATLRCTATSLIPVGPIQWFRGAGPGREL
IYNQKEGHFPRVTTVSDLTKRNNMDFSIRIGNITPADAGTYYCVKFRK
GSPDDVEFKSGAGTELSVR.

Within another embodiment of the invention, the multivalent engager proteins may include a GS linker, a GGS linker, a GGGS linker, a GGGGS linker, a GGGGSGGGGS linker, a GGGGGGGGSGGGGS linker, an Fc hinge region comprising the amino acid sequence LIK, an Fc hinge region comprising the amino acid sequence GLIK, a signal peptide MEFGLSWVFLVAILKGVK, a signal peptide MEFGLSWVFLVAILKGVQ, or a signal peptide MEFGLSWVFLVAILKGVQC.

### 1. Anti-CEACAM6 Antibodies and Antibody Fragments

Within particularly preferred embodiments of the invention the tumor antigen-binding domain of the bispecific T-cell engager is an antibody fragment which recognizes CEACAM6. Representative examples of anti-CEACAM6 antibodies include: BAY1834942 (Tinurilimab), DOS47 (conjugated CEACAM6 antibody), 9A6, 1G2, A1E2, A1E3, 8B7G4, 5E10C7, 4Z3V7, 439424, KOR-SA3544,1H7-4B, EPR4402, EPR4403, EPR4404, TPP-3310. These and other representative anti-CEACAM6 antibodies (and fragments thereof) are disclosed in WO2016150899A2, WO2016150899A3, WO2018070936A1, WO2012040824A1, WO2018128486A1, US20200157214A1, US20200181261A1, US20180162940A1, US20130272958A1, US20190233514A1, WO2018088877A2, CN112409485A, and CN112457402A, all of which are incorporated by reference in their entirety.

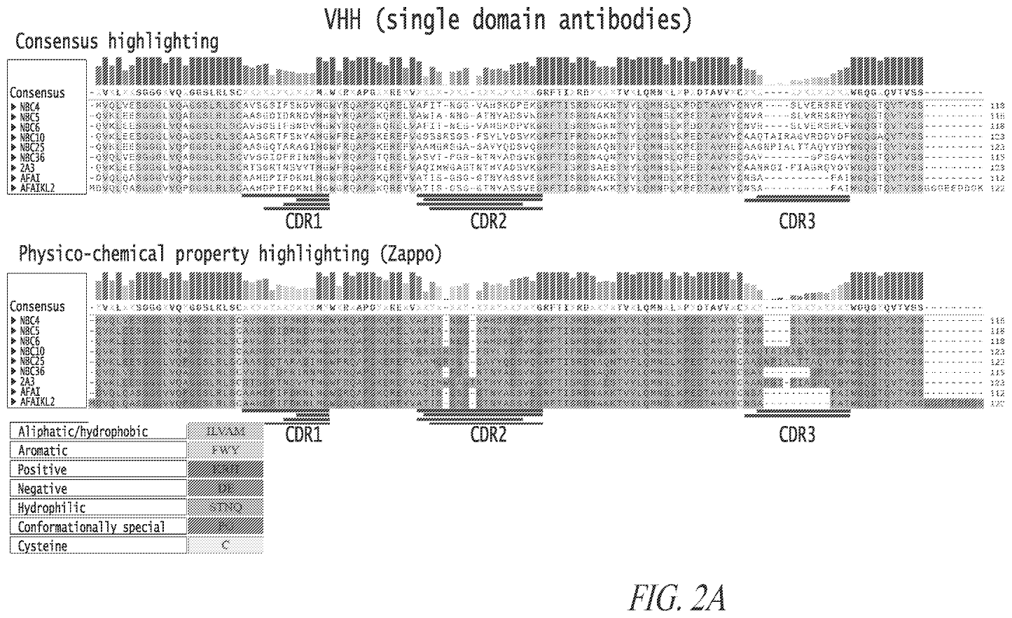

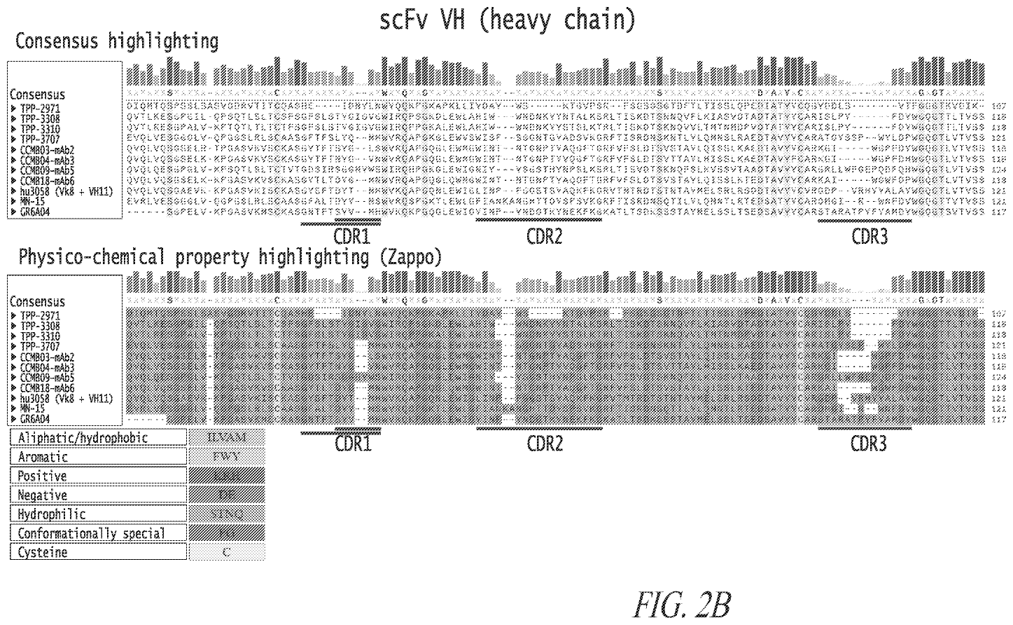

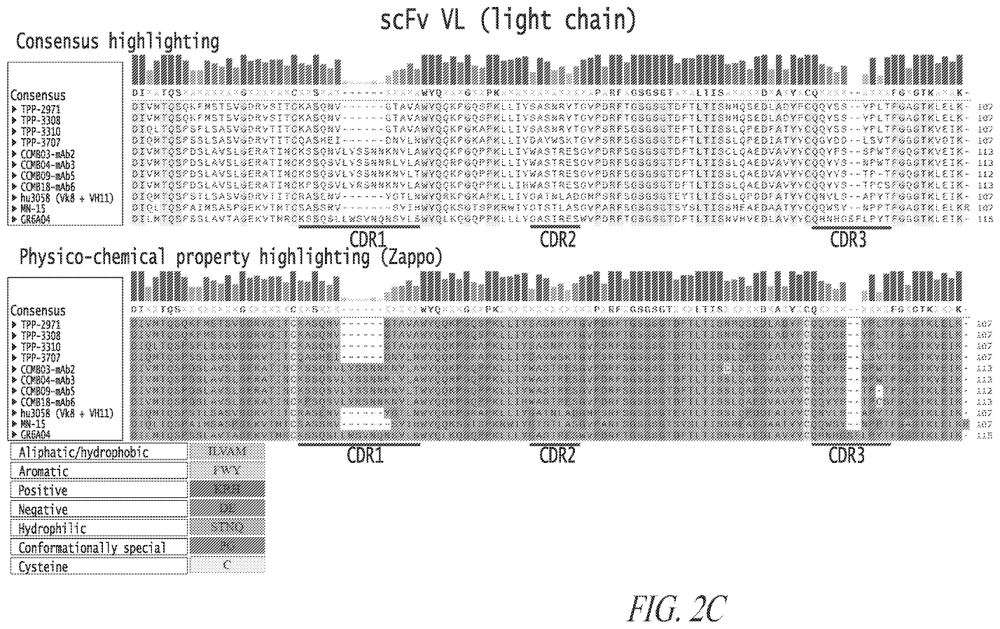

Within certain embodiments of the invention the anti-CEACAM6 antibody or antibody fragment has a sequence comprising a sequence as shown in FIG. 2 (panels 1 or 2). Within other embodiments the anti-CEACAM6 antibody or antibody fragment has a sequence comprising a CDR1, CDR2, or, CDR3 sequence as shown in FIG. 2A (panel 1 or 2), or a sequence as shown in FIG. 2B or 2C (panels 1 or 2).

Within other embodiments of the invention the anti-CEACAM6 antibody or antibody fragment has an overall consensus sequence comprising: X1,X2,V,X3,L,X4,X5,S,G,G,G,X6,V,Q,X7,G,G,S,L,R,L,S,C,X8,X9,X10,X11,X12,X13,X14,X15,X16,X 17,X18,M,X19,W,X20,R,X21,A,P,G,X22,X23,R,E,X24,V,X25,X26,X27,X28,X29,X30,X31,X32,X33, X34,X35,X36,X37,X38,X39,X40,X41,X42,G,R,F,T,I,X43,R,D,X44,X45,X46,X47,T,V,X48,L,Q,M,N, X49,L,X50,P,X51,D,T,A,V,Y,X52,C,X53,X54,X55,X56,X57,X58,X59,X60,X61,X62,X63,X64,X65,X6 6,X67,X68,W,G,Q,G,X69,Q,V,T,V,S,S,X70,X71,X72,X73,X74,X75,X76,X77,X78; wherein the respective amino acids are identified as set forth in Table 1 and Table 2 and Table 2. Within further embodiments of the invention the anti-CEACAM6 antibody or

antibody fragment has a CDR1 consensus sequence comprising: X8,X9,X10,X11,X12,X13,X14,X15,X16,X17,X18,M,X19; or
X9,X10,X11,X12,X13,X14,X15,X16,X17,X18,M,X19; or
X10,X11,X12,X13,X14,X15,X16,X17,X18,M,X19; or X11,X12,X13,X14,X15,X16,X17,X18,M,X19 or X12,X13,X14,X15,X16,X17,X18,M,X19; or X13,X14,X15,X16,X17,X18,M,X19; or
X14,X15,X16,X17,X18,M,X19; or X15,X16,X17,X18,M,X19; or X16,X17,X18,M,X19; wherein the respective amino acids are identified as set forth in Table 1 and Table 2.

Within further embodiments of the invention the anti-CEACAM6 antibody or antibody fragment has a CDR2 consensus sequence comprising:
X25,X26,X27,X28,X29,X30,X31,X32,X33,X34,X35,X36,X37,X38,X39,X40,X41,X42,G; or
X26,X27,X28,X29,X30,X31,X32,X33,X34,X35,X36,X37,X38,X39,X40,X41,X42,G; or
X27,X28,X29,X30,X31,X32,X33,X34,X35,X36,X37,X38,X39,X40,X41,X42,G; or
X25,X26,X27,X28,X29,X30,X31,X32,X33,X34,X35,X36,X37,X38,X39,X40; or
X26,X27,X28,X29,X30,X31,X32,X33,X34,X35,X36,X37,X38,X39,X40; or
X27,X28,X29,X30,X31,X32,X33,X34,X35,X36,X37,X38,X39,X40; or
X25,X26,X27,X28,X29,X30,X31,X32,X33,X34,X35,X36,X37,X38,X39,X40,X41; or
X26,X27,X28,X29,X30,X31,X32,X33,X34,X35,X36,X37,X38,X39,X40,X41; or
X27,X28,X29,X30,X31,X32,X33,X34,X35,X36,X37,X38,X39,X40,X41; or
X25,X26,X27,X28,X29,X30,X31,X32,X33,X34,X35,X36,X37,X38,X39,X40,X41,X42; or
X26,X27,X28,X29,X30,X31,X32,X33,X34,X35,X36,X37,X38,X39,X40,X41,X42; or
X27,X28,X29,X30,X31,X32,X33,X34,X35,X36,X37,X38,X39,X40,X41,X42; wherein the respective amino acids are identified as set forth in Table 1 and Table 2.

Within further embodiments of the invention the anti-CEACAM6 antibody or antibody fragment has a CDR3 consensus sequence comprising:
X55,X56,X57,X58,X59,X60,X61,X62,X63,X64,X65,X66,X67,X68; or
X54,X55,X56,X57,X58,X59,X60,X61,X62,X63,X64,X65,X66,X67,X68; or
X53,X54,X55,X56,X57,X58,X59,X60,X61,X62,X63,X64,X65,X66,X67,X68; wherein the respective amino acids are identified as set forth in Table 1 and Table 2.

TABLE 1

| Physico-chemical properties (Zappo) | Amino acids | Code |
|---|---|---|
| Aliphatic/hydrophobic | ILVAM | Y1 |
| Aromatic | FWY | Y2 |
| Positive | KRH | Y3 |
| Negative | DE | Y4 |
| Hydrophilic | STNQ | Y5 |
| Conformationally special | PG | Y6 |
| Cysteine | C | Y7 |

TABLE 2
X1 is M or no amino acid
X2 is H, Q, or D
X3 is Q or K
X4 is V, E, or Q
X5 is E or A
X6 is L or V
X7 is A or P
X8 is A, V, or R (Y1 or Y3)
X9 is V, A, or T (Y1 or Y5)
X10 is S or H (Y3 or Y5)
X11 is G or D (Y4 or Y6)
X12 is S, D, R, Q, I, or P (Y1 or Y3 or Y4 or Y5 or Y6)
X13 is I, T, or D (Y1 or Y4 or Y5)
X14 is F, D, A, or N (Y1 or Y2 or Y4 or Y5)
X15 is S, R, or D (Y3 or Y4 or Y5)
X16 is N, A, I, V, or K (Y1 or Y3 or Y5)
X17 is D, Y, G, or N (Y2 or Y4 or Y5 or Y6)
X18 is V, A, I, N, T, or L (Y1 or Y5)
X19 is G or H, (Y3 or Y6)
X20 is Y, F, or G
X21 is Q or E
X22 is K or T
X23 is Q or E
X24 is L, F, or Y
X25 is A or G or no amino acid (Y1 or Y6 or no amino acid)
X26 is F, W, S, A, Q, or T or no amino acid (Y1 or Y2 or Y5 or no amino acid)
X27 is I, S, M, or V or no amino acid (Y1 or Y5 or no amino acid)
X28 is T, A, S, G, or M or no amino acid (Y1 or Y5 or Y6 or no amino acid)
X29 is R or W or no amino acid (Y2 or Y3 or no amino acid)
X30 is N, S, P, or G or no amino acid (Y5 or Y6 or no amino acid)
X31 is G, N, E, A, or S or no amino acid (Y1 or Y4 or Y5 or Y6 or no amino acid)
X32 is G, S, A, or R or no amino acid (Y1 or Y3 or Y5 or Y6 or no amino acid)
X33 is T or no amino acid (Y5 or no amino acid)
X34 is V, A, F, S, N, or G or no amino acid (Y1 or Y2 or Y5 or Y6 or no amino acid)
X35 is A, T, or S or no amino acid (Y1 or Y5 or no amino acid)
X36 is H, N, or Y or no amino acid (Y2 or Y3 or Y5 or no amino acid)
X37 is S, Y, or L or no amino acid (Y1 or Y2 or Y5 or no amino acid)
X38 is K, A, V, or Q or no amino acid (Y1 or Y3 or Y5 or no amino acid)
X39 is D or S or no amino acid (Y4 or Y5 or no amino acid)
X40 is P or S or no amino acid (Y5 or Y6 or no amino acid)
X41 is E or V or no amino acid (Y1 or Y4 or no amino acid)
X42 is K, Q, or E or no amino acid (Y3 or Y4 or Y5 or no amino acid)
X43 is S or F
X44 is N or S
X45 is G, A, or D
X46 is K, Q, or E
X47 is N, S, or K
X48 is Y or F
X49 is S or D
X50 is K or Q
X51 is D or E
X52 is Y, H, or S
X53 is N, A, or S or no amino acid (Y1 or Y5 or no amino acid)
X54 is V, A, S, or no amino acid (Y1 or Y5 or no amino acid)
X55 is R, Q, G, Y, N, A, or no amino acid (Y1 or Y2 or Y3 or Y5 or Y6 or no amino acid)
X56 is T, N, R, or no amino acid (Y3 or Y5 or no amino acid)
X57 is A, P, G, or no amino acid (Y1 or Y6 or no amino acid)
X58 is I or no amino acid (Y1 or no amino acid)
X59 is R, A, or no amino acid (Y1 or Y3 or no amino acid)
X60 is S, A, L, P, or no amino acid (Y1 or Y5 or Y6 or no amino acid)
X61 is L, G, T, I, or no amino acid (Y1 or Y5 or Y6 or no amino acid)
X62 is V, T, A, or no amino acid (Y1 or Y5 or no amino acid)
X63 is E, R, A, G, or no amino acid (Y1 or Y3 or Y4 or Y6 or no amino acid)
X64 is R, D, Q, P, or no amino acid (Y3 or Y4 or Y5 or Y6 or no amino acid)
X65 is S, D, Y, Q, or no amino acid (Y2 or Y4 or Y5 or no amino acid)
X66 is R, Y, G, F, or no amino acid (Y2 or Y3 or Y6 or no amino acid)
X67 is E, D, A, or no amino acid (Y1 or Y4 or no amino acid)
X68 is Y, F, I, or no amino acid (Y1 or Y2 or no amino acid)
X69 is T or A
X70 is G or no amino acid
X71 is G or no amino acid
X72 is G or no amino acid
X73 is E or no amino acid
X74 is E or no amino acid
X75 is D or no amino acid
X76 is D or no amino acid
X77 is G or no amino acid
X78 is K or no amino acid

Within other embodiments of the invention the anti-CEACAM6 antibody or antibody fragment has a VH consensus sequence comprising:
X30,X31,X32,X33,X34,X35,X36 or X25,X26,X27,X28,X29,X30,X31,X32,X33,X34,X35,X36; a VH CDR2 consensus sequence comprising:
X48,X49,X50,X51,X52,X53,X54,X55,X56,X57,X58,X59,X60,X61,X62,X63,X64,X65,X66; and/or a VH CDR3 consensus sequence comprising:
X94,X95,X96,X97,X98,X99,X100,X101,X102,X103,X104,X105,X106,X107; wherein the respective amino acids are identified as set forth in Table 1 and Table 3.

TABLE 3
X25 is H, G, or no amino acid (Y3 or Y6 or no amino acid)
X26 is E, F, Y, D, N, or no amino acid (Y2 or Y4 or Y5 or no amino acid)
X27 is S, T, A, or no amino acid (Y1 or Y5 or no amino acid)
X28 is L, F, I, or no amino acid (Y1 or Y2 or no amino acid)
X29 is S, T, R, or no amino acid (Y3 or Y5 or no amino acid)
X30 is T, L, S, N, D, or no amino acid (Y1 or Y4 or Y5 or no amino acid)
X31 is I, Y, G, or no amino acid (Y1 or Y2 or Y6 or no amino acid)
X32 is D, G, Q, T, Y, V, or no amino acid (Y1 or Y2 or Y4 or Y5 or Y6 or no amino acid)
X33 is N, I, H, or no amino acid (Y1 or Y3 or Y5 or no amino acid)
X34 is Y, G, or no amino acid (Y2 or Y6 or no amino acid)
X35 is L, V, M, W, or no amino acid (Y1 or Y2 or no amino acid)
X36 is N, G, H, S, or no amino acid (Y3 or Y5 or Y6 or no amino acid)
X48 is Y, H, W, N, L, F, or no amino acid (Y1 or Y2 or Y3 or Y5 or no amino acid)
X49 is D, I, or no amino acid (Y1 or Y4 or no amino acid)
X50 is A, W, S, N, Y, or no amino acid (Y1 or Y2 or Y5 or no amino acid)
X51 is Y, F, T, P, N, or no amino acid (Y2 or Y5 or Y6 or no amino acid)
X52 is K or no amino acid (Y3 or no amino acid)
X53 is A or no amino acid (Y1 or no amino acid)
X54 is W, S, N, Y, F, or no amino acid (Y2 or Y5 or no amino acid)
X55 is S, N, G, T, or no amino acid (Y5 or Y6 or no amino acid)
X56 is D, G, H, or no amino acid (Y3 or Y4 or Y6 or no amino acid)
X57 is N, S, T, G, or no amino acid (Y5 or Y6 or no amino acid)
X58 is K, T, P, or no amino acid (Y3 or Y5 or Y6 or no amino acid)
X59 is Y, G, T, H, S, D, K, or no amino acid (Y2 or Y3 or Y4 or Y5 or Y6 or no amino acid)
X60 is Y or no amino acid (Y2 or no amino acid)
X61 is K, N, S, A, V, or no amino acid (Y1 or Y3 or Y5 or no amino acid)
X62 is T, D, Q, P, E, or no amino acid (Y4 or Y5 or Y6 or no amino acid)
X63 is G, A, S, K, or no amino acid (Y1 or Y3 or Y5 or Y6 or no amino acid)
X64 is V, L, F, or no amino acid (Y1 or Y2 or no amino acid)
X65 is P, K, T, or no amino acid (Y3 or Y5 or Y6 or no amino acid)
X66 is S, T, G, or no amino acid (Y5 or Y6 or no amino acid)
X94 is Y, I, A, K, G, D, S, or no amino acid (Y1 or Y2 or Y3 or Y4 or Y5 or Y6 or no amino acid)
X95 is D, S, T, G, R, A, M, or no amino acid (Y1 or Y3 or Y4 or Y5 or Y6 or no amino acid)
X96 is D, L, G, I, P, A, or no amino acid (Y1 or Y4 or Y6 or no amino acid)
X97 is L, P, Y, I, R, or no amino acid (Y1 or Y2 or Y3 or Y6 or no amino acid)
X98 is S, Y, W, A, or no amino acid (Y1 or Y2 or Y5 or no amino acid)
X99 is S, F, V, T, or no amino acid (Y1 or Y2 or Y5 or no amino acid)
X100 is P, G, R, or no amino acid (Y3 or Y6 or no amino acid)
X101 is E, H, Y, or no amino acid (Y2 or Y3 or Y4 or no amino acid)
X102 is W, P, Y, F, or no amino acid (Y2 or Y6 or no amino acid)
X103 is W, G, Q, Y, or no amino acid (Y2 or Y5 or Y6 or no amino acid)
X104 is Y, P, D, W, A, N, or no amino acid (Y1 or Y2 or Y4 or Y5 or Y6 or no amino acid)
X105 is F, L, M, or no amino acid (Y1 or Y2 or no amino acid)
X106 is V, D, Q, A, or no amino acid (Y1 or Y4 or Y5 or no amino acid)
X107 is T, Y, P, H, V, or no amino acid (Y1 or Y2 or Y3 or Y5 or Y6 or no amino acid)

Within other embodiments of the invention the anti-CEACAM6 antibody or antibody fragment has a VL CDR1 consensus sequence comprising: X17,X18,S, X19,X20, X21,X22,X23,X24,X25,X26,X27,X28,X29,X30,X31,X32; a VL CDR2 consensus sequence comprising: X42,X43,X44,X45,X46,X47,X48; and/or a VL CDR3 consensus sequence comprising: Q,X64,X65,X66,X67,X68,X69,X70,X71,X72,X73; wherein the respective amino acids are identified as set forth in Table 1 and Table 4.

TABLE 4
X17 is K, Q, R, S, or no amino acid (Y3 or Y5 or no amino acid)
X18 is A, S, P, or no amino acid (Y1 or Y5 or Y6 or no amino acid)
X19 is Q, H, E, S, or no amino acid (Y3 or Y4 or Y5 or no amino acid)
X20 is N, E, S, R, or no amino acid (Y3 or Y4 or Y5 or no amino acid)
X21 is V, I, L, or no amino acid (Y1 or no amino acid)
X22 is L or no amino acid (Y1 or no amino acid)
X23 is Y, W, or no amino acid (Y2 or no amino acid)
X24 is S, R, or no amino acid (Y3 or Y5 or no amino acid)
X25 is S, V, or no amino acid (Y1 or Y5 or no amino acid)
X26 is N or no amino acid (Y5 or no amino acid)
X27 is N, Q, or no amino acid (Y5 or no amino acid)
X28 is G, D, K, R, Y, N, or no amino acid (Y2 or Y3 or Y4 or Y5 or Y6 or no amino acid)
X29 is T, N, L, G, S, or no amino acid (Y1 or Y5 or Y6 or no amino acid)
X30 is A, Y, T, or no amino acid (Y1 or Y2 or Y5 or no amino acid)
X31 is V, L, I, or no amino acid (Y1 or no amino acid)
X32 is A, N, T, H, S, or no amino acid (Y1 or Y3 or Y5 or no amino acid)
X42 is S, D, W, or G (Y2 or Y4 or Y5 or Y6)
X43 is A or T (Y1 or Y5)
X44 is S, Y, or T (Y2 or Y5)
X45 is N, W, T, or I (Y1 or Y2 or Y5)
X46 is R, S, or L (Y1 or Y3 or Y5)
X47 is Y, K, E, or A (Y1 or Y2 or Y3 or Y4)
X48 is T, S, or D (Y4 or Y5)
X64 is Q, G, N, H, or no amino acid (Y3 or Y5 or Y6 or no amino acid)
X65 is Y, V, W, N, or no amino acid (Y1 or Y2 or Y5 or no amino acid)
X66 is S, D, F, Y, L, H, or no amino acid (Y1 or Y2 or Y3 or Y4 or Y5 or no amino acid)
X67 is S, D, Y, G, or no amino acid (Y2 or Y4 or Y5 or Y6 or no amino acid)
X68 is S or no amino acid (Y5 or no amino acid)
X69 is F or no amino acid (Y2 or no amino acid)
X70 is Y, L, S, N, T, A, or no amino acid (Y1 or Y2 or Y5 or no amino acid)
X71 is P, S, or no amino acid (Y5 or Y6 or no amino acid)
X72 is L, V, W, C, Y, P, or no amino acid (Y1 or Y2 or Y6 or Y7 or no amino acid)
X73 is T, S, or no amino acid (Y5 or no amino acid)

In certain embodiments, the single-domain antibody, antibody fragment, or antigen-binding fragment thereof is humanized.

Within preferred embodiments of the invention the expression and release of the immune cell engager should be transient and tumor-specific to minimize toxicity caused by leakage and inadvertent tagging of healthy cells surrounding the tumor. Use of a broadly tumor-associated antigen that is not necessarily restricted to tumor cells (such as TfR1 or GLUT1) by the immune cell engager facilitates the destruction of multiple cell types in the tumor microenvironment, potentially even disrupting tumor structural components that are comprised of non-cancer cells and enhancing immune cell infiltration, ultimately resulting in improved anti-tumor immune activation and tumor clearance. Any antibody or antibody fragment used for the antigen-binding domain of the immune cell engager should also avoid internalization and lysosomal degradation and efficiently decorate the target cell surface.

In some embodiments, the two domains of the immune cell engager are joined by a flexible linker, of which many variations exist and are known to those skilled in the art. In one embodiment, multiple tandem repeats of the sequence GGGGS (SEQ ID NO:4) are used as linkers. In other embodiments, any suitable linker known in the art may be used, e.g. the flexible hinge region of Fc.

### B. Oncolytic Viruses

As noted above, the present invention provides oncolytic viruses which may be engineered to express and secrete an immune cell engager protein. Briefly, an “oncolytic virus” is a virus that is capable of replicating in and killing tumor cells. Within certain embodiments the virus can be engineered in order to more selectively target tumor cells. Representative examples of oncolytic viruses include without limitation, adenovirus, coxsackievirus, H-1 parvovirus, herpes simplex virus (HSV), influenza virus, measles virus, Myxoma virus, Newcastle disease virus, parvovirus picornavirus, reovirus, rhabdovirus (e.g. vesicular stomatitis virus (VSV)), paramyxovirus such as Newcastle disease virus, picornavirus such as poliovirus or Seneca valley virus, pox viruses such as vaccinia virus (e.g. Copenhagen, Indiana Western Reserve, and Wyeth strains), reovirus, or retrovirus such as murine leukemia virus. Further representative examples are described in: U.S. Pat. Nos. 8,147,822 and 9,045,729 (oncolytic rhabdovirus/VSV); U.S. Pat. No. 9,272,008 (oncolytic Measles virus); U.S. Pat. Nos. 7,223,593, 7,537,924, 7,063,835, 7,063,851, 7,118,755, 8,216,564, 8,277,818, and 8,680,068 (oncolytic herpes virus vectors); and U.S. Pat. No. 8,980,246 (oncolytic vaccinia virus), all of which are incorporated by reference in their entirety.

Within certain embodiments of the invention, the oncolytic virus is Herpes Simplex virus (e.g., HSV-1 or HSV-2). Briefly, Herpes Simplex Virus (HSV) 1 and 2 are members of the Herpesviridae family, which infects humans. The HSV genome contains two unique regions, which are designated unique long (U_{L}) and unique short (U₅) region. Each of these regions is flanked by a pair of inverted terminal repeat sequences. There are about 75 known open reading frames. The viral genome has been engineered to develop oncolytic viruses for use in e.g. cancer therapy. Tumor-selective replication of HSV may be conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic. Tumor selective replication of HSV may also be conferred by controlling expression of key viral genes such as ICP27 and/or ICP4.

Suitable oncolytic HSV may be derived from either HSV-1 or HSV-2, including any laboratory strain or clinical isolate. In some embodiments, the oHSV may be or may be derived from one of laboratory strains HSV-1 strain 17, HSV-1 strain F, or HSV-2 strain HG52. In other embodiments, it may be of or derived from non-laboratory strain JS-1. Other suitable HSV-1 viruses include HrrR3 (Goldstein and Weller, J. Virol. 62, 196-205, 1988), G207 (Mineta et al. Nature Medicine. 1 (9): 938-943, 1995; Kooby et al. The FASEB Journal, 13 (11): 1325-1334, 1999); G47Delta (Todo et al. Proceedings of the National Academy of Sciences. 2001; 98 (11): 6396-6401); HSV 1716 (Mace et al. Head & Neck, 2008; 30 (8): 1045-1051; Harrow et al. Gene Therapy. 2004; 11 (22): 1648-1658); HF10 (Nakao et al. Cancer Gene Therapy. 2011; 18 (3): 167-175); NV1020 (Fong et al. Molecular Therapy, 2009; 17 (2): 389-394); T-VEC (Andtbacka et al. Journal of Clinical Oncology, 2015: 33 (25): 2780-8); J100 (Gaston et al. PloS one, 2013; 8 (11): e81768); M002 (Parker et al. Proceedings of the National Academy of Sciences, 2000; 97 (5): 2208-2213); NV1042 (Passer et al. Cancer Gene Therapy. 2013; 20 (1): 17-24); G207-IL2 (Carew et al. Molecular Therapy, 2001; 4 (3): 250-256); rQNestin34.5 (Kambara et al. Cancer Research, 2005; 65 (7): 2832-2839); G47Δ-mIL-18 (Fukuhara et al. Cancer Research, 2005; 65 (23): 10663-10668); and those vectors which are disclosed in PCT applications PCT/US2017/030308 entitled “HSV Vectors with Enhanced Replication in Cancer Cells”, and PCT/US2017/018539 entitled “Compositions and Methods of Using Stat⅓ Inhibitors with Oncolytic Herpes Virus”, all of the above of which are incorporated by reference in their entirety.

As disclosed herein, the oHSV vector may include one or more immune cell engager (ICE) or bispecific T-cell engager (BITE) expression cassettes that include genes or nucleotide sequences that encode the BiTE fusion protein under the control of a heterologous promoter. In one embodiment, the bispecific T-cell engager expression cassette carried by the oncolytic HSV is controlled by the strong constitutive EF1α promoter and uses a UCHT1 antibody for the CD3-targeting arm and an anti-CEACAM6 antibody for the tumor-targeting arm. In another embodiment, the bispecific T-cell engager expression cassette is controlled by a tumor-specific promoter. In yet another embodiment, the CD3-targeting arm of the BiTE is selected from the following antibodies: UCHT1, SP34, or CRIS-7. In a further embodiment, the tumor-targeting arm of the BiTE is comprised of an scFv or nanobody designed to bind one of the following tumor-associated antigens: B7-H3, B7-H6, CD70, CEA, CEACAM6, CSPG4, EGFRvIII, EphA2, EpCAM, EGFR, ErbB2 (HER2), FAP, FRα, GD2, GD3, HLA-A1+MAGE1, IL-11Rα, IL-13Rα2, Lewis-Y, Mesothelin, Muc1, Muc16, NKG2D, PSMA, ROR1, TAG72, VEGFR2, TfR1, GLUT1, or PD-L1.

The oHSV vector may have at least one γ34.5 gene that is modified with miRNA target sequences in its 3′ UTR as disclosed herein; there are no unmodified γ34.5 genes in the vector. In some embodiments, the oHSV has two modified γ34.5 genes; in other embodiments, the oHSV has only one γ34.5 gene, and it is modified. In some embodiments, the modified γ34.5 gene(s) are constructed in vitro and inserted into the oHSV vector as replacements for the viral gene(s). When the modified γ34.5 gene is a replacement of only one γ34.5 gene, the other γ34.5 is deleted. Either native γ34.5 gene can be deleted. Alternatively, both copies of the native γ34.5 gene can be deleted. In one embodiment, the terminal repeat, which comprises γ34.5 gene and ICP4 gene, is deleted. As discussed herein, the modified γ34.5 gene may comprise additional changes, such as having an exogenous promoter.

The oHSV may have additional mutations, which may include disabling mutations (e.g., deletions, substitutions, insertions), which may affect the virulence of the virus or its ability to replicate. For example, mutations may be made in any one or more of ICP6, ICPO, ICP4, ICP27, ICP47, ICP24, ICP56, ICP34.5, and LAT. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible upon delivery of an inducer or inducible upon a cellular event or particular environment. In other embodiments, the promoter of a viral gene may be substituted with a promoter of a different viral gene that reduces the risk of recombination event in the repetitive and non-unique region of the native, or natural, promoter.

In certain embodiments, the expression of ICP4 or ICP27 is controlled by an exogenous (i.e., heterologous) promoter, e.g., a tumor-specific promoter. Exemplary tumor-specific promoters include survivin, CEA, CXCR4, PSA, ARR2PB, or telomerase; other suitable tumor-specific promoters may be specific to a single tumor type and are known in the art. Other elements may also be present. In some cases, an enhancer such as NFKB/oct4/sox2 enhancer is present. As well, the 5′UTR may be exogenous, such as a 5′UTR from growth factor genes such as FGF.

The oHSV may also have genes and nucleotide sequences that are non-HSV in origin. For example, a sequence that encodes a prodrug, a sequence that encodes a cytokine or other immune stimulating factor, a tumor-specific promoter, an inducible promoter, an enhancer, a sequence homologous to a host cell, among others may be in the oHSV genome. Exemplary sequences encode IL12, IL15, IL15 receptor alpha subunit, OX40L, PD-L1 blocker or a PD-1 blocker. For sequences that encode a product, they are operatively linked to a promoter sequence and other regulatory sequences (e.g., enhancer, polyadenylation signal sequence) necessary or desirable for expression.

The regulatory region of viral genes may be modified to comprise response elements that affect expression. Exemplary response elements include response elements for NF-κB, Oct-¾-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

Within certain embodiments of the invention the oncolytic Herpes Virus is as described in PCT/US2022/021798, which is incorporated by reference in its entirety.

### C. Specific Herpes Virus Constructs-VG301

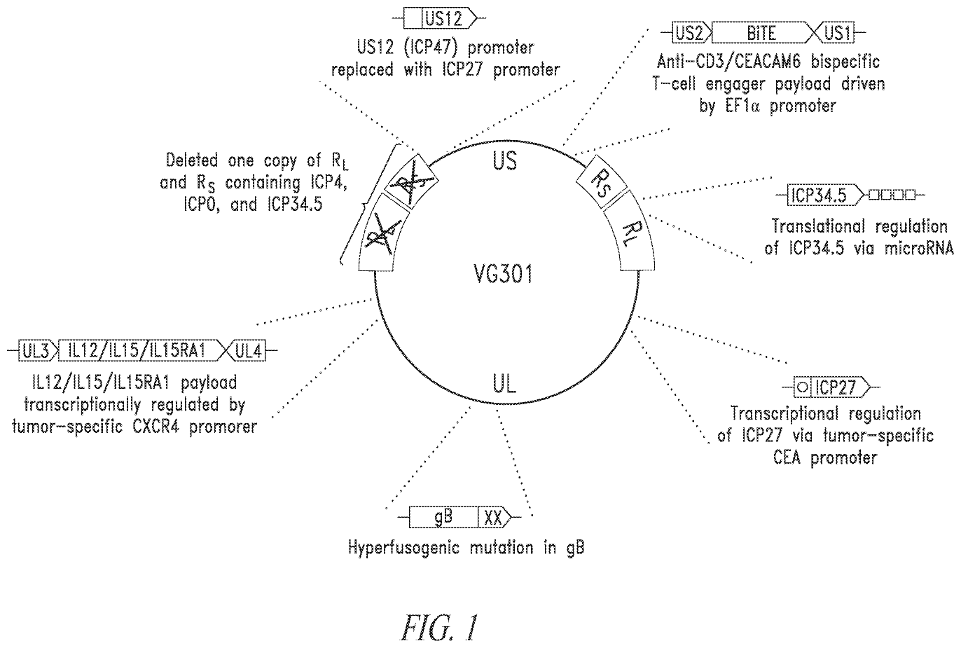

One preferred construct of the invention is provided in FIG. 1. Briefly, FIG. 1 diagrammatically depicts the overall structural organization of the double-stranded deoxyribonucleic acid (DNA) elements of VG301 (also referred to herein as “VG21306”). “CXCR4” means C—X—C Motif Chemokine Receptor 4; “; EF1α” means elongation factor 1-alpha; “gB” means glycoprotein B; “ICP” means infected cell protein; “IL” means interleukin; “R_{L}” means repeat long; “RNA” means ribonucleic acid; “R₅” means repeat short; “UL” means unique long; “US” means unique short.

VG301 is a recombinant HSV-1 platform that utilizes both transcriptional and translational dual-regulation of key viral genes to limit virus replication to tumor cells and enhance tumor-specific virulence without compromising safety. In addition, VG301 expresses a payload cassette composed of IL12, IL15 and IL15 alpha receptor subunit. The payload expression is under control of the CXCR4 promoter for expression in tumor cells. Finally, a viral glycoprotein B(gB) in VG301 was truncated to facilitate virus spread in the tumor by enhanced fusogenicity.

### D. Post-Transcriptional Regulation

In VG301, ICP34.5 expression is post-transcriptionally regulated. Briefly, in wild-type HSV-1, there are 2 copies of the ICP34.5 gene. In VG301, one copy of ICP34.5 has been deleted. For the remaining ICP34.5 gene, VG301 inserts multiple copies of binding domains for miR124 (also referred to as miR-124) and miR143 (also referred to as miR-143) in the 3′UTR region to regulate its expression post-transcriptionally.

ICP34.5 is encoded by the HSV late gene g-34.5. It is well known for its function of suppressing anti-viral immunity of host cells, particularly neuronal cells, to cause neurotoxicity. To abolish the functions of ICP34.5 in neurons and other normal cells while remaining its activity in tumor cells for robust replication, instead of deleting the gene or using a specific promoter to control the expression of ICP34.5 to target specific tumors, VG301 uses microRNAs as a post-transcriptional control to achieve differential expression of ICP34.5 in tumor cells. Briefly, miRNAs are ˜22 nucleotides, noncoding small RNAs coded by miRNA genes, which are transcribed by RNA polymerase II to produce primary miRNA (pri-miRNA). Mature single-stranded (ss) miRNA forms the miRNA-associated RNA-induced silencing complex (miRISC). miRNA in miRISC may influence gene expression by binding to the 3′-untranslated region (3′-UTR) in the target mRNA. This region consists of sequences recognized by miRNA. If the complementarity of the miRNA: mRNA complex is perfect, the mRNA is degraded by Ago2, a protein belonging to the Argonaute family. However, if the complementarity is not perfect, the translation of the target mRNA is suppressed.

miRNAs are expressed differentially in a tissue specific fashion. One of the examples is miR-124. While the precursors of miR-124 from different species are different, the sequences of mature miR-124 in human, mice, rats are completely identical. MIR-124 is the most abundantly expressed miRNA in neuronal cells and is highly expressed in the immune cells and organs (Qin et al., 2016, miRNA-124 in immune system and immune disorders. Frontiers in Immunology, 7 (OCT), 1-8). Another example of differential expression of miRNA is miR143 (Lagos-Quintana et al., 2002, Identification of tissue-specific MicroRNAs from mouse. Current Biology, 12 (9), 735-739. MiR-143 is constitutively expressed in normal tissues but significantly downregulated in cancer cells (Michael et al., 2003, Reduced Accumulation of Specific MicroRNAs in Colorectal Neoplasia. Molecular Cancer Research, 1 (12), 882-891.

The 3′ UTR region of ICP34.5 gene in VG301 contains multiple copies of binding sites that are completely complementary to miR124 (binding site SEQ ID NO:6) and miR143 (binding site SEQ ID NO:7). Binding of miR-124 and miR-143 to the 3′UTR of ICP34.5 mRNA causes degradation of the mRNA; therefore the gene is post-transcriptionally downregulated in normal cells but not tumor cells. This design allows differential expression of ICP34.5 in tumor cells.

In one embodiment, five tandem copies of binding sites for both miR-124 and miR-143 are inserted into the 3′-UTR of ICP34.5. In other embodiments, any suitable number of miR binding sites in any suitable arrangement may be used. In certain embodiments, the miR binding sites may be separated by a region of spacer DNA of any suitable length. In preferred embodiments, the length of the spacer DNA ranges from 1 bp to 27 bp. Within further embodiments there is no spacer which separates miR binding sites.

### E. Expression of ICP27 IN VG301 Is Transcriptionally Controlled

HSV-1 viral replication depends on a cascade of expression of viral genes, with expression of immediate early genes (particularly ICP4 and ICP27) controlling subsequent expression of viral early genes and late genes that govern the lytic replication cycle of the virus. Deletion of ICP4 or ICP27 results in complete abrogation of viral replication and a significant reduction in viral gene expression, which makes ICP4 and ICP27 excellent targets for tumor specific regulation in oncolytic HSV.

While ICP4 is a major transcriptional factor for viral gene expression, ICP27 is a multi-functional protein that regulates transcription of many virus genes. ICP27 functions in all stages of mRNA biogenesis from transcription, RNA processing and export through to translation. ICP27 has also been implicated in nuclear protein quality control, cell cycle control, activation of stress signaling pathways and prevention of apoptosis.

In VG301, the native promoter of ICP27 is replaced with a 432 bp promoter for human carcinoembryonic antigen (CEA) (SEQ ID NO:13) to facilitate virus replication in CEA positive tumor cells.

### F. Payload Expression of VG301 is Enhanced

VG301 co-expresses IL12 (SEQ ID NO:9), IL15 (SEQ ID NO:10) and IL15 receptor alpha subunit (SEQ ID NO:11) to further stimulate an immunomodulatory response. Expression of IL12 promotes polarization of antigen exposed T cells towards an inflammatory and anti-tumor T_{H}1 phenotype, while IL-15 activates NK cells to further increase tumor killing and activation of antigen presenting cells. In addition to IL15 expression, VG301 encodes IL15Ra to further enhance immune stimulation.

Transcription of IL-12, IL-15, and IL-15Ra is driven by a single tumor-specific promoter (CXCR4) (SEQ ID NO:8) and the polypeptides are linked with 2A self-cleaving peptides (Z. Liu et al., 2017, Systemic comparison of 2A peptides for cloning multi-genes in a polycistronic vector. Scientific Reports, 7 (1), 1-9) (SEQ ID NO:12) that generate the 3 individual proteins through a mechanism of ribosomal skipping during translation.

### G. Truncated Glycoprotein B (Gb)

HSV-1 fusion is a crucial step of infection. It is dependent on four essential viral glycoproteins (gB, gD, gH, and gL), which mediate entry into host cells by merging the viral envelope with a host cell membrane. The core fusion protein is glycoprotein B (gB), a 904-residue glycosylated transmembrane protein encoded by the UL27 gene of HSV-1. Multiple types of mutations within the cytoplasmic domain of gB have yielded a hyperfusogenic phenotype, increasing cell-cell fusion (Chowdary & Heldwein, 2010, Syncytial Phenotype of C-Terminally Truncated Herpes Simplex Virus Type 1 gB is Associated with Diminished Membrane Interactions. Journal of Virology, 84 (10), 4923-4935. In one embodiment, gB may be modified by truncating C-terminal amino acids 877 to 904 from the full-length protein.

### H. Modified ICP47 Promoter

In the HSV genome, the promoter controlling expression of the US12 gene, which encodes ICP47, is identical to the promoter controlling expression of the US1 gene, which is located approximately 13k base pairs from the US12 gene. In addition, large regions of the native ICP47 promoter include repetitive sequences that may facilitate spurious homologous recombination events. Thus, replacement of the native ICP47 promoter with a heterologous (e.g., exogenous) promoter is predicted to improve viral genomic stability.

In HSV, both ICP27 and ICP47 are encoded by immediate early genes, expressed very early after infection, and share many regulatory elements. To reduce the risk of homologous recombination while maintaining a natural expression pattern, the VG301 construct replaces the native ICP47 promoter with the ICP27 promoter.

In some embodiments, the native ICP27 promoter includes the entire sequence of DNA located between the coding regions of U_{L}53 (gK) and U_{L}54 (ICP27). In one embodiment, the ICP27 promoter includes the 538 bp sequence set forth in SEQ ID NO:5.

In other embodiments, the ICP27 promoter sequence may be 90%, 80%, 70%, 60%, or 50% identical to the ICP27 promoter sequence of any known human herpes virus 1 strain, e.g., human herpes virus 1 strain 17 (NCBI reference sequence NC_001806.2).

Specific modifications to wild type-HSV-1 strain 17+ are set forth below in Table 5.

TABLE 5
Genetic Modification in VG301 from wild type HSV-1, strain 17+

| | | Modification | |
|---|---|---|---|
| Modification | Modification Type | Location | Function |
| Deletion of both | Deletion | Terminal repeat | Wild type HSV-1 contains two |
| terminal repeat long | | long (TR_{L}) and | identical copies of TR_{L} and TR_{S}, |
| (TR_{L}) and short (TR_{S}) | | short (TR_{S}) | with the second copy designated |
| regions containing | | | as IR_{L} and IR_{S}. Removal of one |
| ICP4, ICP0, ICP34.5 | | | copy may attenuate virulence |
| and LAT genes | | | while freeing up genomic space |
| | | | for payload insertion. |
| Replacement of | Replacement | ICP27 gene | To facilitate virus replication in |
| native ICP27 | | promoter | CEA positive tumor cells |
| promoter with CEA | | | |
| promoter | | | |
| Insertion of 5x miR- | Insertion | ICP34.5 gene 3′- | To inhibit expression of ICP34.5 in |
| 124 binding site and | | UTR | cells with high expression of miR- |
| miR-143 binding site | | | 124 and/or miR-143 |
| in the ICP34.5 3′-UTR | | | |
| Deletion of 84 bp in | Deletion | 3′ end of (gB) | To make HSV-1 gB protein |
| 3′ end of | | coding region | truncated by 28 amino acids at its |
| glycoprotein B (gB) | | | c-terminus for enhanced |
| coding region | | | fusogenicity |
| Insertion of IL-12, IL- | Insertion | Between UL3 | To facilitate expression of IL-12, |
| 15, and IL-15Rα as an | | and UL4 genes | IL-15, and IL-15Rα in all types of |
| expression cassette | | | infected cells that do not |
| with CXCR4 promoter | | | suppress CXCR4 promoter |
| | | | function |
| Replacement of | Replacement | ICP47 gene | To reduce the risk of |
| native ICP47 | | promoter | recombination events in the |
| promoter with ICP27 | | | repetitive and non-unique ICP47 |
| promoter | | | promoter region |
| Insertion of anti- | Insertion | Between US1 | To facilitate robust expression of |
| CD3/CEACAM6 | | and US2 genes | secretable anti-CD3/CEACAM6 |
| bispecific T-cell | | | bispecific T-cell engager (BiTE) in |
| engager (BiTE) | | | a broad range of cell types |
| expression cassette | | | |
| with EF1α promoter | | | |

CEA = carcinoembryonic antigen; CXCR4 = C-X-C Motif Chemokine Receptor 4; gB = glycoprotein B; HSV-1 = herpes simplex virus-1; ICP0 = infected cell polypeptide 0; ICP27 = infected cell polypeptide 27; ICP47 = infected cell polypeptide 47; ICP34.5 = infected cell polypeptide 34.5; IL = interleukin; miR = microRNA; Ra = receptor alpha; TR_{L} = terminal repeat long; TR_{S} = terminal repeat short; IR_{L} = internal repeat long; IR_{S} = internal repeat short; UL = unique long; US = unique short; LAT = latency-associated transcript; CD3 = cluster of differentiation 3; CEACAM6 = carcinoembryonic antigen-related cell adhesion molecule 6; EF1α = elongation factor 1-alpha; BiTE = bispecific T-cell engager. VG21306, VG21324, and VG22305 are historical names for VG301 variants that use UCHT, SP34, or CRIS7, respectively, as the antibody targeting CD3.

VG301 is designed to take advantage of Applicant's existing transcription and translation dual regulated (TTDR) OV platform to increase tumor specificity and enhance patient safety without sacrificing virus replication capability. The TTDR platform incorporates transcriptional regulation of the key HSV gene transactivator ICP27 via the tumor-specific CEA promoter and translational regulation of the major neurovirulence determinant ICP34.5 via inclusion of tandem microRNA binding sites in the 3′-UTR of ICP34.5 with the binding sites comprising multiple copies of DNA sequences that are complementary to microRNAs which are present at relatively high concentrations in normal cells but are downregulated in cancer cells. As a result, VG301 is aimed at tumors that express CEA in addition to the TAA targeted by the BiTE due to the CEA-mediated regulation of ICP27 expression. In some alternative embodiments, the native ICP27 promoter is deleted and replaced with a different tumor-specific promoter such as the RAN, CEA, or CXCR4 promoter.

Within further embodiments of the invention short hairpin RNA (shRNA) mediated gene silencing may be utilized to reduce or eliminate expression of the broadly expressed tumor associated antigen (i.e., the second tumor-associated antigen) such as TfR1 from infected cells in order to extend the length of productive viral infection by protecting infected cells from premature death via retargeted immune cells. To that end, shRNA targeting the second tumor-associated antigen (e.g., TfR1 or GLUT1) may also be expressed by the same oncolytic virus that is engineered to express the immune cell engager protein.

Because cells infected with oncolytic virus are already destined for destruction, in some embodiments short hairpin RNA (shRNA) mediated gene silencing may be utilized to reduce or eliminate expression of the broadly expressed tumor associated antigen (i.e., the second tumor-associated antigen) such as TfR1 from infected cells in order to extend the length of productive viral infection by protecting infected cells from premature death via retargeted immune cells. To that end, shRNA targeting the second tumor-associated antigen (e.g., TfR1 or GLUT1) may also be expressed by the same oncolytic virus that is engineered to express the immune cell engager protein.

As an alternative to using an oncolytic virus to express and secrete the immune cell engager protein, in some embodiments the immune cell engager protein may be encoded within an mRNA molecule that is encapsulated within lipid nanoparticles and injected into the tumor for internalization and translation. Tumor-specific expression of the immune cell engager protein from said mRNA may be achieved by adding miRNA target sequences to the 3′-end and/or the 5′-end of said mRNA, wherein said miRNA target sequences are recognized by miRNAs that are less abundant in the targeted tumor cells compared to normal cells.

Antibody binding to TfR1 normally triggers internalization and eventual destruction of the antibody. To overcome this problem, in certain embodiments, the immune cell engager proteins of the present invention may include all or a portion of the recycling anti-TfR1 monoclonal antibody H7 (see https://doi.org/10.1080/19420862.2018.1564510, which is incorporated by reference in its entirety), which can dissociate from its bound antigen in the acidic environment of the sorting endosomes and avoid lysosomal degradation, while recycling back to be displayed on the plasma membrane (Neiveyans 2019).

1. Alternative Compositions and Methods related to Immune Cell Engager Proteins

As noted herein, the present invention provides a variety of Immune Cell Engager Proteins. Within one embodiment of the invention, isolated nucleic acid molecules are provided which encode the Immune Cell Engager Proteins. As utilized herein the term “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

The Immune Cell Engager Proteins of the present invention may also be contained within an Expression Cassette. As used herein the term “Expression Cassette” is meant to include any type of genetic construct containing a nucleic acid coding for a gene product in which part or all of the nucleic acid encoding sequence is capable of being transcribed. Within preferred embodiments the expression cassette comprises a promoter which is operably linked to a nucleic acid sequence encoding the immune cell engager protein.

Within further embodiments of the invention the expression cassette is introduced into a vector that facilitate entry into a host cell and maintenance of the expression cassette in the host cell. Numerous such vectors are commonly used and well known to those of skill in the art, including for example, those which are available from Invitrogen, Stratagene, Clontech and others.

### J. Therapeutic Compositions

Therapeutic compositions are provided that may be used to prevent, treat, or ameliorate the effects of a disease, such as, for example, cancer. More particularly, therapeutic compositions are provided comprising at least one oncolytic virus and/or immune cell engager protein as described herein.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus or immune cell engager protein and that is not toxic to the subject to whom it is administered (see generally Remington: The Science and Practice of Pharmacy, Lippincott Williams & Wilkins; 21st ed. (May 1, 2005 and in The United States PharmacopE1A: The National Formulary (USP 40-NF 35 and Supplements).

In the case of an oncolytic virus and immune cell engager proteins as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethylene glycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oHSV to a cancer cell will preferably not induce an immune response in the individual (subject) receiving the composition (and will preferably be administered without undue toxicity).

The compositions provided herein can be provided at a variety of concentrations. For example, dosages of oncolytic virus can be provided which range from about 10⁴ pfu to about 10¹⁰ pfu. Within further embodiments, the dosage can range from about 10⁶ pfu to about 10⁷ pfu, or from about 10⁷ pfu to about 10⁸ pfu, or from about 10⁸ pfu to 10⁹ pfu, and may be administered as a single dose or as multiple doses spread out over time. Doses may be administered daily, weekly, biweekly, monthly, or bimonthly, and dosage frequency may be cyclical, with each cycle comprising a repeating dosage pattern (e. g. once a week or biweekly dose administration for about 4 weeks comprising one cycle, repeating for up to about 24 cycles). Within other embodiments of the invention, the virus can be provided in ranges from about 5×10⁴ pfu/kg to about 2×10⁹ pfu/kg for intravenous delivery in humans. For intratumoral injection, the preferred dosage can range from about 10⁶ pfu to about 10⁹ pfu per dose (with an injectable volume which ranges from about 0.1 mL to about 5 mL).

Within certain embodiments of the invention, lower or higher dosages than standard may be utilized. Hence, within certain embodiments less than about 10⁶ pfu or more than about 10⁹ pfu can be administered to a patient.

Within certain embodiments of the invention, lower dosages than standard may be utilized. Hence, within certain embodiments less than about 10⁶ pfu/ml (with up to 4 ml being injected into a patient every 2-3 weeks) can be administered to a patient.

The compositions may be stored at a temperature conducive to stable shelf-life and includes room temperature (about 20° C.), 4° C., −20° C., −80° C., and in liquid N₂. Because compositions intended for use in vivo generally do not have preservatives, storage will generally be at colder temperatures. Compositions may be stored dry (e.g., lyophilized) or in liquid form.

### K. Administration

In addition to the compositions described herein, various methods of using such compositions to treat or ameliorate cancer are provided, comprising the step of administering an effective dose or amount of an oncolytic virus or immune cell engager protein as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The compositions are used to treat cancer. The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemias, lymphomas, myelomas and sarcomas. Representative forms of leukemias include acute myeloid leukemia (AML) and representative forms of lymphoma include B cell lymphomas. Further examples include, but are not limited to cancer of the bile duct, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemias and lymphomas), kidney, larynx, lung, liver, oral cavity, ovaries, pancreas, prostate, skin (e.g., melanoma and squamous cell carcinoma), GI (e.g., esophagus, stomach, and colon) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy). Benign tumors and other conditions of unwanted cell proliferation may also be treated.

Particularly preferred cancers to be treated include those with high levels of CEACAM6 expression. Representative examples include lung tumors, cervical, ovarian (e.g., mucinous adenocarcinomas), breast and prostate tumors, glioblastomas, tumors of the gastro-intestinal tract (and associated organs) e.g., esophagus, cholangiocarcinoma, anal, stomach, intestine, pancreatic, colon and liver, and all surface injectable tumors (e.g., melanomas).

The recombinant oncolytic viruses described herein may be given by a route that is, for example, oral, topical, parenteral, systemic, intravenous, intramuscular, intraocular, intrathecal, intratumoral, subcutaneous, or transdermal. Within certain embodiments the oncolytic virus may be delivered by a cannula, by a catheter, or by direct injection. The site of administration may be intra-tumor or at a site distant from the tumor. The route of administration will often depend on the type of cancer being targeted.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as radiotherapy or chemotherapy using, e.g., a chemotherapeutic agent such as etoposide, ifosfamide, adriamycin, vincristine, doxycycline, and others.

Recombinant herpes simplex viruses described herein may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional.

A therapeutically effective amount is preferably administered. This is an amount that is sufficient to show benefit to the subject. The actual amount administered, and the time-course of administration will depend at least in part on the nature of the cancer, the condition of the subject, site of delivery, and other factors.

Within yet other embodiments of the invention the oncolytic virus can be administered by a variety of methods, e.g., intratumorally, intravenously, or, after surgical resection of a tumor.

The following are some exemplary numbered embodiments of the present disclosure.

1. A recombinant herpes simplex virus comprising a modified oncolytic herpes virus genome, wherein the modified herpes virus genome comprises an expression cassette encoding an immune cell engager protein, wherein the immune cell engager protein comprises a T-cell binding domain and a tumor-associated antigen binding domain.

2. The recombinant herpes simplex virus of embodiment 1, wherein the T-cell binding domain comprises an anti-CD3, anti-CD28, anti-OX40, or anti-4-1BB antibody or antibody fragment thereof. Within one embodiment the antibody or antibody fragment is humanized.

3. The recombinant herpes simplex virus of embodiment 2, wherein the anti-CD3 antibody or fragment thereof is selected from the group consisting of UCHT1 (SEQ ID NO:1), SP34 (SEQ ID NO:2), and CRIS-7 (SEQ ID NO:3).

4. The recombinant herpes simplex virus of embodiment 1, wherein the tumor-associated antigen binding domain comprises an antibody or fragment thereof. Within one embodiment the antibody or antibody fragment is humanized.

5. The recombinant herpes simplex virus of embodiment 4, wherein the antibody or fragment thereof comprises a scFv or a nanobody.

6. The recombinant herpes simplex virus of embodiment 4, wherein the antibody or fragment thereof specifically binds to a tumor-associated antigen selected from the group consisting of B7-H3, B7-H6, CD70, CEA, CSPG4, EGFRvIII, EphA2, EpCAM, EGFR, ErbB2 (HER2), FAP, FRα, GD2, GD3, HLA-A1+MAGE1, IL-11Rα, IL-13Rα2, Lewis-Y, Mesothelin, Muc1, Muc16, NKG2D, PSMA, ROR1, TAG72, VEGFR2, TfR1, GLUT1, or PD-L1.

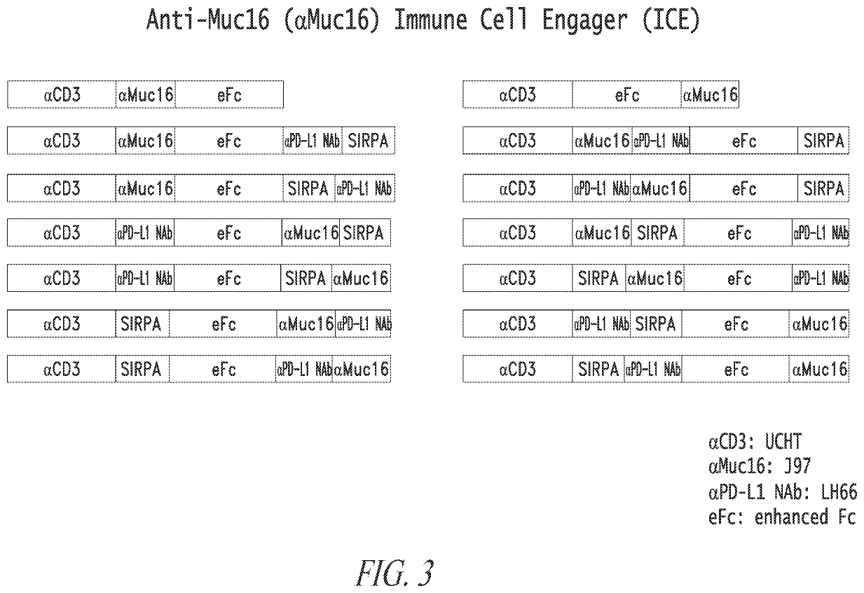

7. The recombinant herpes simplex virus of embodiment 4, wherein the tumor-associated antigen is CEACAM6, Muc16, PD-L1, or TfR1. Within certain embodiments of the invention the anti-CEACAM6 antibody or antibody fragment has a sequence comprising a sequence as shown in FIG. 2 (panels 1 or 2). Within other embodiments the anti-CEACAM6 antibody or antibody fragment has a sequence comprising a CDR1, CDR2, or, CDR3 sequence as shown in FIG. 2A (panel 1 or 2), or a sequence as shown in FIG. 2B or 2C (panels 1 or 2). Within certain embodiments of the invention the tumor-associated antigen is shown in FIG. 3, FIG. 10, and FIG. 13.

Within other embodiments of the invention the anti-CEACAM6 antibody or antibody fragment has an overall consensus sequence comprising:
X1,X2,V,X3,L,X4,X5,S,G,G,G,X6,V,Q,X7,G,G,S,L,R,L,S,C,X8,X9,X10,X11,X12,X13,X14,X15,X16,X 17,X18,M,X19,W,X20,R,X21,A,P,G,X22,X23,R,E,X24,V,X25,X26,X27,X28,X29,X30,X31,X32,X33, X34,X35,X36,X37,X38,X39,X40,X41,X42,G,R,F,T,I,X43,R,D,X44,X45,X46,X47,T,V,X48,L,Q,M,N, X49,L,X50,P,X51,D,T,A,V,Y,X52,C,X53,X54,X55,X56,X57,X58,X59,X60,X61,X62,X63,X64,X65,X6 6,X67,X68,W,G,Q,G,X69,Q,V,T,V,S,S,X70,X71,X72,X73,X74,X75,X76,X77,X78; wherein the respective amino acids are identified as set forth in Table 1 and Table 2.

Within further embodiments of the invention the anti-CEACAM6 antibody or antibody fragment has a CDR1 consensus sequence comprising:
or X8,X9,X10,X11,X12,X13,X14,X15,X16,X17,X18,M,X19; X9,X10,X11,X12,X13,X14,X15,X16,X17,X18,M,X19; or
X10,X11,X12,X13,X14,X15,X16,X17,X18,M,X19; or X11,X12,X13,X14,X15,X16,X17,X18,M,X19
or X12,X13,X14,X15,X16,X17,X18,M,X19; or X13,X14,X15,X16,X17,X18,M,X19; or
X14,X15,X16,X17,X18,M,X19; or X15,X16,X17,X18,M,X19; or X16,X17,X18,M,X19; wherein the respective amino acids are identified as set forth in Table 1 and Table 2.

Within yet other embodiments of the invention the anti-CEACAM6 antibody or antibody fragment has a CDR2 consensus sequence comprising:
X25,X26,X27,X28,X29,X30,X31,X32,X33,X34,X35,X36,X37,X38,X39,X40,X41,X42,G; or
X26,X27,X28,X29,X30,X31,X32,X33,X34,X35,X36,X37,X38,X39,X40,X41,X42,G; or
X27,X28,X29,X30,X31,X32,X33,X34,X35,X36,X37,X38,X39,X40,X41,X42,G; or
X25,X26,X27,X28,X29,X30,X31,X32,X33,X34,X35,X36,X37,X38,X39,X40; or
X26,X27,X28,X29,X30,X31,X32,X33,X34,X35,X36,X37,X38,X39,X40; or
X27,X28,X29,X30,X31,X32,X33,X34,X35,X36,X37,X38,X39,X40; or
X25,X26,X27,X28,X29,X30,X31,X32,X33,X34,X35,X36,X37,X38,X39,X40,X41; or
X26,X27,X28,X29,X30,X31,X32,X33,X34,X35,X36,X37,X38,X39,X40,X41; or
X27,X28,X29,X30,X31,X32,X33,X34,X35,X36,X37,X38,X39,X40,X41; or
X25,X26,X27,X28,X29,X30,X31,X32,X33,X34,X35,X36,X37,X38,X39,X40,X41,X42; or
X26,X27,X28,X29,X30,X31,X32,X33,X34,X35,X36,X37,X38,X39,X40,X41,X42; or
X27,X28,X29,X30,X31,X32,X33,X34,X35,X36,X37,X38,X39,X40,X41,X42; wherein the respective amino acids are identified as set forth in Table 1 and Table 2.

Within further embodiments of the invention the anti-CEACAM6 antibody or antibody fragment has a CDR3 consensus sequence comprising:
X55,X56,X57,X58,X59,X60,X61,X62,X63,X64,X65,X66,X67,X68; or
X54,X55,X56,X57,X58,X59,X60,X61,X62,X63,X64,X65,X66,X67,X68; or
X53,X54,X55,X56,X57,X58,X59,X60,X61,X62,X63,X64,X65,X66,X67,X68; wherein the respective amino acids are identified as set forth in Table 1 and Table 2.

Within other embodiments of the invention the anti-CEACAM6 antibody or antibody fragment has a VH CDR1 consensus sequence comprising:
X30,X31,X32,X33,X34,X35,X36 or X25,X26,X27,X28,X29,X30,X31,X32,X33,X34,X35,X36; a VH CDR2 consensus sequence comprising:
X48,X49,X50,X51,X52,X53,X54,X55,X56,X57,X58,X59,X60,X61,X62,X63,X64,X65,X66; and/or a VH CDR3 consensus sequence comprising:
X94,X95,X96,X97,X98,X99,X100,X101,X102,X103,X104,X105,X106,X107; respective amino acids are identified as set forth in Table 1 and Table 3. wherein the

Within other embodiments of the invention the anti-CEACAM6 antibody or antibody fragment has a VL CDR1 consensus sequence comprising:
CDR2 X17,X18,S,X19,X20,X21,X22,X23,X24,X25,X26,X27,X28,X29,X30,X31,X32; a VL consensus sequence comprising: X42,X43,X44,X45,X46,X47,X48; and/or a VL CDR3 consensus sequence comprising: Q,X64,X65,X66,X67,X68,X69,X70,X71,X72,X73; wherein the respective amino acids are identified as set forth in Table 1 and Table 4.

8. The recombinant herpes simplex virus of embodiment 1, wherein the T-cell binding domain and the tumor-associated antigen binding domain are joined by a flexible linker comprising one or more repeats of a peptide comprising the amino acid sequence GGGGS (SEQ ID NO:4).

9. The recombinant herpes simplex virus of embodiment 1, wherein the expression cassette comprises an EF1α promoter controlling expression of the immune cell engager protein.

10. The recombinant herpes simplex virus of embodiment 1, wherein the immune cell engager protein is capable of being secreted from a host cell infected by the recombinant herpes simplex virus.

11. The recombinant herpes simplex virus of embodiment 1, further comprising at least one miRNA target sequence operably linked to a first copy of an ICP34.5 gene, and a second copy of the ICP34.5 gene comprises an inactivating mutation.

12. The recombinant herpes simplex virus of embodiment 11, wherein the mutation is deletion of one terminal repeat long region and/or one terminal repeat short region of the viral genome.

13. The recombinant herpes simplex virus of embodiment 11, comprising from two to ten miRNA target sequences operably linked to the first copy of the ICP34.5 gene.

14. The recombinant herpes simplex virus of embodiment 11, wherein the miRNA target sequences are inserted into a 3′ untranslated region of the first copy of the ICP34.5 gene.

15. The recombinant herpes simplex virus of embodiment 13, wherein the from two to ten miRNA target sequences bind at least two different miRNAs.

16. The recombinant herpes simplex virus of embodiment 15, wherein the miRNAs target sequences are miR-124 (SEQ ID NO:6) and miR-143 (SEQ ID NO:7).

17. The recombinant herpes simplex virus of embodiment 1, wherein the modified herpes virus genome comprises additional mutations or modifications in viral genes ICP4, ICPO, ICP27, ICP47, and/or LAT.

18. The recombinant herpes simplex virus of embodiment 17, wherein said virus is modified by replacing one or more native viral promoters with a heterologous promoter.

19. The recombinant herpes simplex virus of embodiment 18, wherein the modification is replacement of the entire promoter-regulatory region of ICP27 with a tumor specific promoter.

20. The recombinant herpes simplex virus of embodiment 19, wherein the ICP27 promoter is replaced with a CEA promoter (SEQ ID NO:13).

21. The recombinant herpes simplex virus of embodiment 18, wherein the ICP47 promoter is replaced by a ICP27 promoter (SEQ ID NO:5).

22. The recombinant herpes simplex virus of embodiment 1, further comprising at least one nucleic acid encoding a non-viral protein selected from the group consisting of immunostimulatory factors, antibodies, and checkpoint blocking peptides, wherein the at least one nucleic acid is operably linked to a generic or a tumor-specific promoter.

23. The recombinant herpes simplex virus of embodiment 22, wherein the non-viral protein is selected from the group consisting of IL12 (SEQ ID NO:9), IL15 (SEQ ID NO:10), and IL15 receptor alpha subunit (SEQ ID NO:11).

24. The recombinant herpes simplex virus of embodiment 22, wherein the tumor-specific promoter is promoter is CRCX4 (SEQ ID NO:8).

25. The recombinant herpes simplex virus of embodiment 1, further comprising a nucleic acid sequence encoding a fusogenic form of glycoprotein B.

26. The recombinant herpes simplex virus of embodiment 25, wherein the glycoprotein B can be truncated with a deletion occurring after amino acid 876.

27. The recombinant herpes simplex virus of any one of embodiments 1 to 26, wherein the oncolytic herpes virus is HSV-1.

28. A immune cell engager protein comprising a T-cell binding domain and a tumor-associated antigen binding domain, wherein said tumor-associated antigen binding domain is anti-CEACAM6, anti-Muc16, anti-PD-L1, or anti-TfR1. Within certain embodiments of the invention the antibody or antibody fragment is an anti-CEACAM6 antibody or antibody fragment that has a sequence comprising a sequence as shown in FIG. 2A (panel 1 or 2). Within other embodiments the anti-CEACAM6 antibody or antibody fragment has a sequence comprising a CDR1, CDR2, or, a CDR3 sequence as shown in FIG. 2A (panel 1 or 2).

Within other embodiments of the invention the anti-CEACAM6 antibody or antibody fragment has an overall consensus sequence comprising:
X1,X2,V,X3,L,X4,X5,S,G,G,G,X6,V,Q,X7,G,G,S,L,R,L,S,C,X8,X9,X10,X11,X12,X13,X14,X15,X16,X 17,X18,M,X19,W,X20,R,X21,A,P,G,X22,X23,R,E,X24,V,X25,X26,X27,X28,X29,X30,X31,X32,X33, X34,X35,X36,X37,X38,X39,X40,X41,X42,G,R,F,T,I,X43,R,D,X44,X45,X46,X47,T,V,X48,L,Q,M,N, X49,L,X50,P,X51,D,T,A,V,Y,X52,C,X53,X54,X55,X56,X57,X58,X59,X60,X61,X62,X63,X64,X65,X6 6,X67,X68,W,G,Q,G,X69,Q,V,T,V,S,S,X70,X71,X72,X73,X74,X75,X76,X77,X78; wherein the respective amino acids are identified as set forth in Table 1 and Table 2.

Within further embodiments of the invention the anti-CEACAM6 antibody or antibody fragment has a CDR1 consensus sequence comprising: X8,X9,X10,X11,X12,X13,X14,X15,X16,X17,X18,M,X19; or
X9,X10,X11,X12,X13,X14,X15,X16,X17,X18,M,X19; or X10,X11,X12,X13,X14,X15,X16,X17,X18,M,X19; or X11,X12,X13,X14,X15,X16,X17,X18,M,X19 or X12,X13,X14,X15,X16,X17,X18,M,X19; or X13,X14,X15,X16,X17,X18,M,X19; or X14,X15,X16,X17,X18,M,X19; or X15,X16,X17,X18,M,X19; or X16,X17,X18,M,X19; wherein the respective amino acids are identified as set forth in Table 1 and Table 2.

Within yet other embodiments of the invention the anti-CEACAM6 antibody or antibody fragment has a CDR2 consensus sequence comprising:
X25,X26,X27,X28,X29,X30,X31,X32,X33,X34,X35,X36,X37,X38,X39,X40,X41,X42,G; or
X26,X27,X28,X29,X30,X31,X32,X33,X34,X35,X36,X37,X38,X39,X40,X41,X42,G; or
X27,X28,X29,X30,X31,X32,X33,X34,X35,X36,X37,X38,X39,X40,X41,X42,G; or
X25,X26,X27,X28,X29,X30,X31,X32,X33,X34,X35,X36,X37,X38,X39,X40; or
X26,X27,X28,X29,X30,X31,X32,X33,X34,X35,X36,X37,X38,X39,X40; or
X27,X28,X29,X30,X31,X32,X33,X34,X35,X36,X37,X38,X39,X40; or
X25,X26,X27,X28,X29,X30,X31,X32,X33,X34,X35,X36,X37,X38,X39,X40,X41; or
X26,X27,X28,X29,X30,X31,X32,X33,X34,X35,X36,X37,X38,X39,X40,X41; or
X27,X28,X29,X30,X31,X32,X33,X34,X35,X36,X37,X38,X39,X40,41; or
X25,X26,X27,X28,X29,X30,X31,X32,X33,X34,X35,X36,X37,X38,X39,X40,X41,X42; or
X26,X27,X28,X29,X30,X31,X32,X33,X34,X35,X36,X37,X38,X39,X40,X41,X42; or
X27,X28,X29,X30,X31,X32,X33,X34,X35,X36,X37,X38,X39,X40,X41,X42; wherein the respective amino acids are identified as set forth in Table 1 and Table 2.

Within further embodiments of the invention the anti-CEACAM6 antibody or antibody fragment has a CDR3 consensus sequence comprising:
X55,X56,X57,X58,X59,X60,X61,X62,X63,X64,X65,X66,X67,X68; or
X54,X55,X56,X57,X58,X59,X60,X61,X62,X63,X64,X65,X66,X67,X68; or X53,X54,X55,X56,X57,X58,X59,X60,X61,X62,X63,X64,X65,X66,X67,X68; wherein the respective amino acids are identified as set forth in Table 1 and Table 2.

29. The immune cell engager protein according to embodiment 28, wherein said T-Cell binding domain is an anti-CD3, anti-CD28, anti-OX40, or an anti-4-1BB antibody or antibody fragment.

30. The immune cell engager protein according to embodiment 29 wherein said anti-CD3 antibody or fragment thereof is selected from the group consisting of UCHT1 (SEQ ID NO:1), SP34 (SEQ ID NO:2), and CRIS-7 (SEQ ID NO:3).

31. The immune cell engager protein according to embodiment 28 wherein said T-cell binding domain and the tumor-associated antigen binding domain are joined by a flexible linker.

32. The immune cell engager protein according to embodiment 31 wherein said flexible linker comprises one or more repeats of a peptide comprising the amino acid sequence GGGGS (SEQ ID NO:4).

33. An isolated nucleic acid molecule encoding an immune cell engager protein according to any one of embodiments 28 to 32.

34. An expression vector which directs the expression of the nucleic acid sequence according to embodiment 33.

35. A method for inhibiting tumor cells, comprising providing a therapeutically effective amount of recombinant herpes simplex virus according to any one of embodiments 1 to 27, the immune cell engager protein according to any one of embodiments 28 to 32 or the expression vector according to embodiment 34.

36. A pharmaceutical composition comprising the recombinant herpes simplex virus according to any one of embodiments 1 to 27, an immune cell engager protein according to embodiments 28 to 32, or an expression vector according to embodiment 34, and a pharmaceutically acceptable carrier.

37. A method for treating cancer in a subject suffering therefrom, comprising the step of administering to a subject a therapeutically effective amount of the pharmaceutical composition according to embodiment 36.

38. The method according to embodiment 37 wherein said pharmaceutical composition is administered intravenously, or, injected directly into a tumor.

The following Examples are offered by way of illustration and not by way of limitation.

### EXAMPLES

### Example 1

### Construction and Characterization of Novel Anti-Muc16, Anti-PD-L1, and Anti-TfR Immune Cell Engagers

Objective: Immune cell engagers (ICE) are designed to redirect and activate immune effector cells, utilizing at least one arm to target one or more tumor-associated antigens and another arm or multiple arms directed against one or more activating receptors in immune effector cells. The present invention describes and characterizes novel immune cell engagers comprising a plurality of functional domains engineered to target widely expressed tumor-associated antigens. Off-target effects can be minimized by delivering the immune cell engager intratumorally either by direct injection of the purified engager protein, by injecting RNA encoding the immune cell engager (as LNP-encapsulated mRNA, self-amplifying RNA, etc.), or by delivering the engager as a secretable payload expressed by an oncolytic virus such as an oncolytic herpesvirus.

Study design: A panel of 14 different immune cell engagers targeting cells expressing Muc16 was engineered as depicted in FIG. 3. Another panel of 7 different immune cell engagers targeting cells expressing PD-L1 was engineered as depicted in FIG. 10. Two additional immune cell engagers targeting cells expressing TfR were engineered as depicted in FIG. 13. Said engagers comprise a plurality of functional domains, with each domain joined to adjacent domains using linker polypeptides such as the common linker amino acid sequence GGGGS or variants thereof. The order in which the functional domains are arranged differs between each engager, and the name of each engager is comprised of the names of each functional domain in the order in which they appear and separated by dashes. The functional domains comprise one or more domains designed to activate an effector cell either by binding to CD3 on the effector cell using an antibody or antibody fragment such as the anti-CD3 (αCD3) antibody UCHT, or by displaying an Fc region such as enhanced Fc (eFc) that is recognized by Fc-gamma receptors on the effector cell. The functional domains further comprise one or more domains that bind to one or more antigens expressed on a target cell, such as the anti-Muc16 (αMuc16) antibody J97, the anti-PD-L1 (αPD-L1) antibody LH56, or the anti-TfR (αTfR) antibody H7. The functional domains may optionally comprise one or more domains that bind to a negative regulator of effector cell function, such as the anti-PD-L1 (αPD-L1) neutralizing antibody (NAb) LH66 or a fragment of SIRPa that is recognized by the regulatory protein CD47.

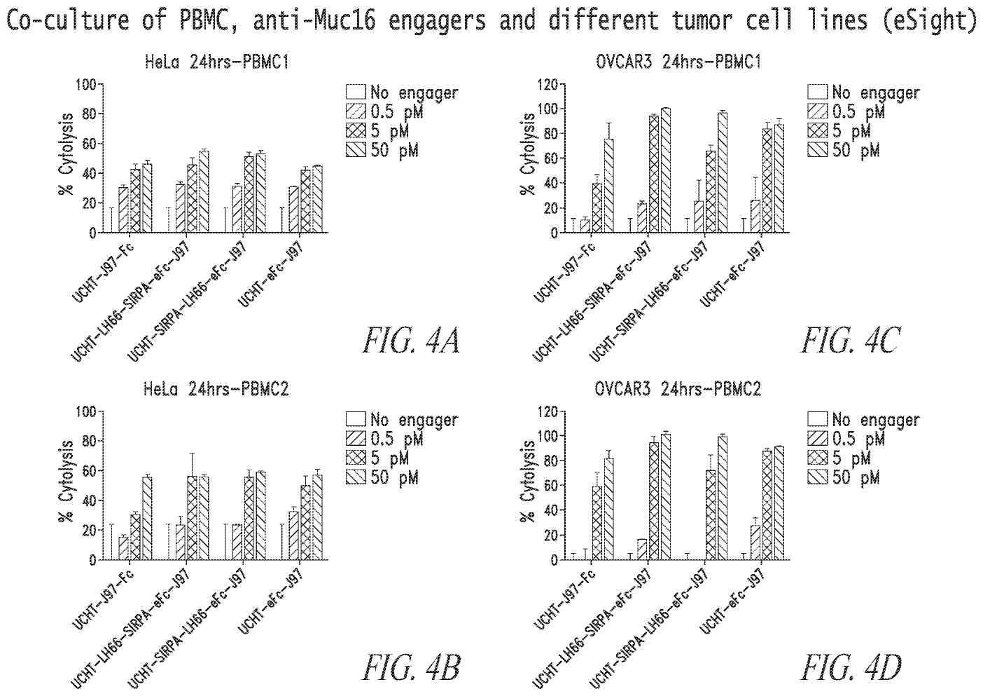

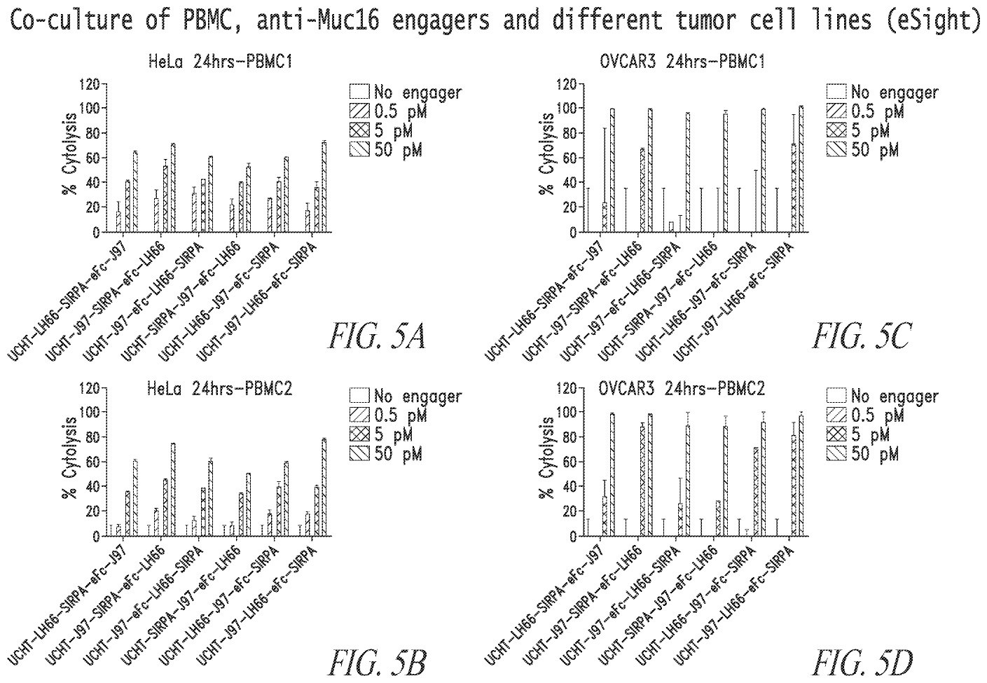

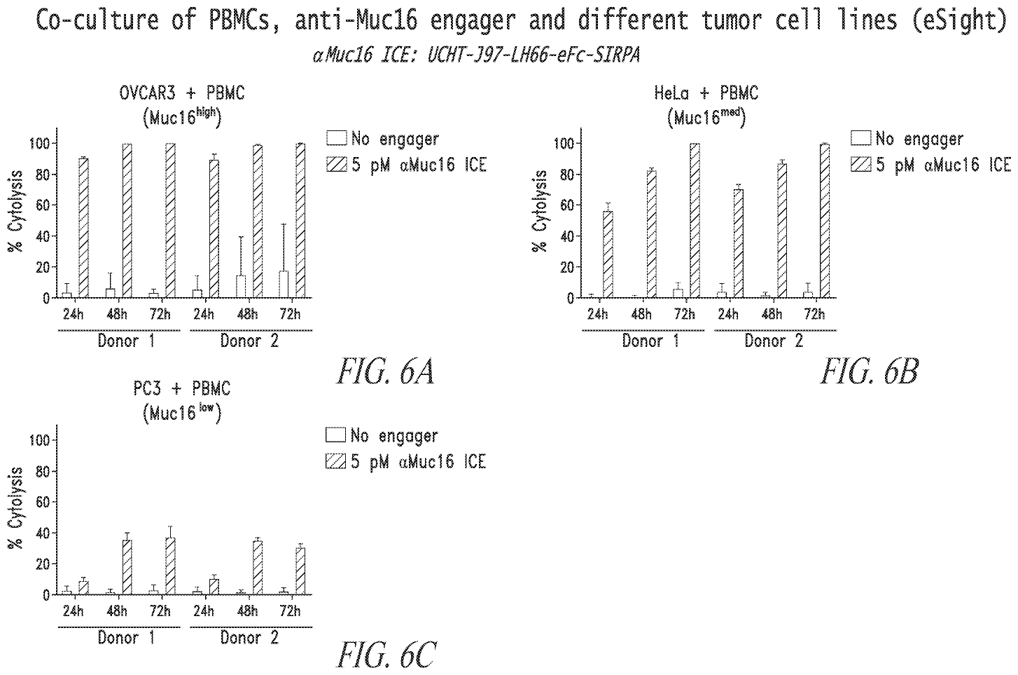

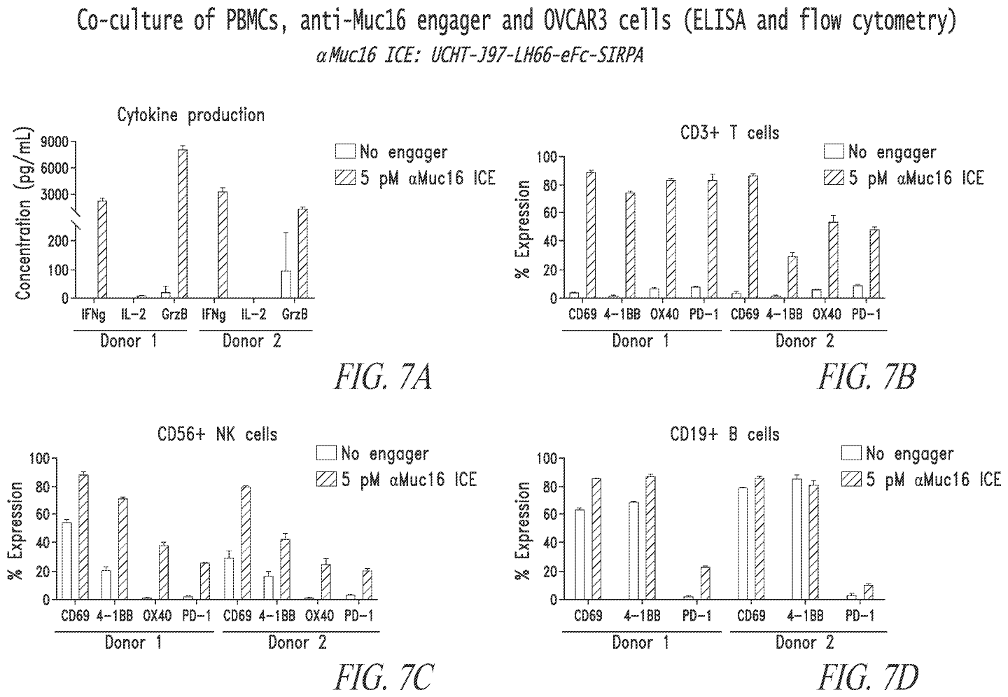

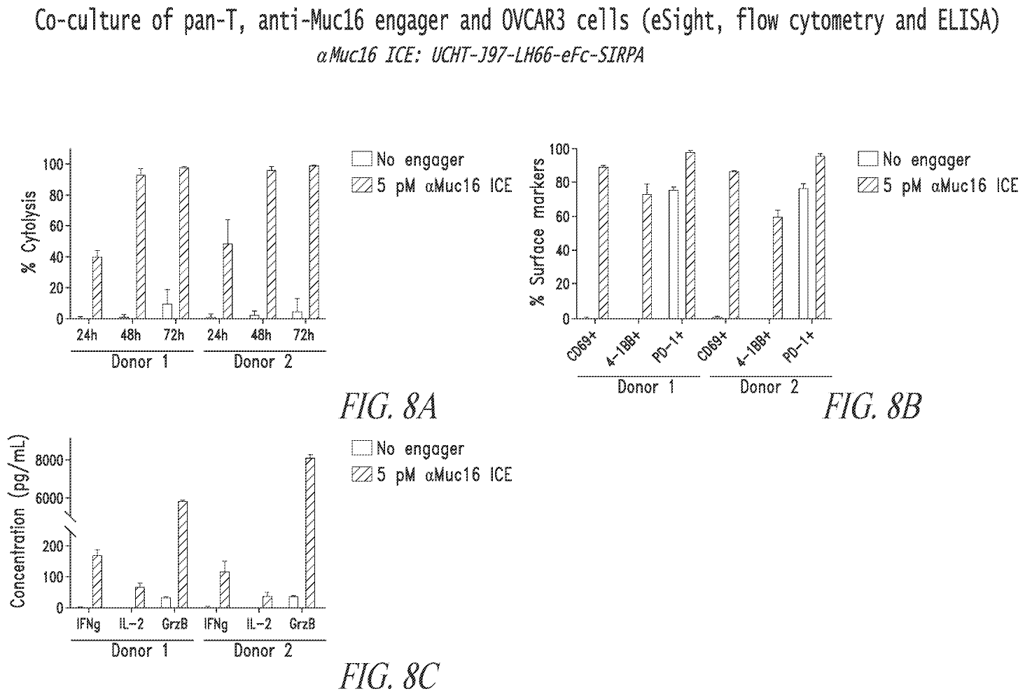

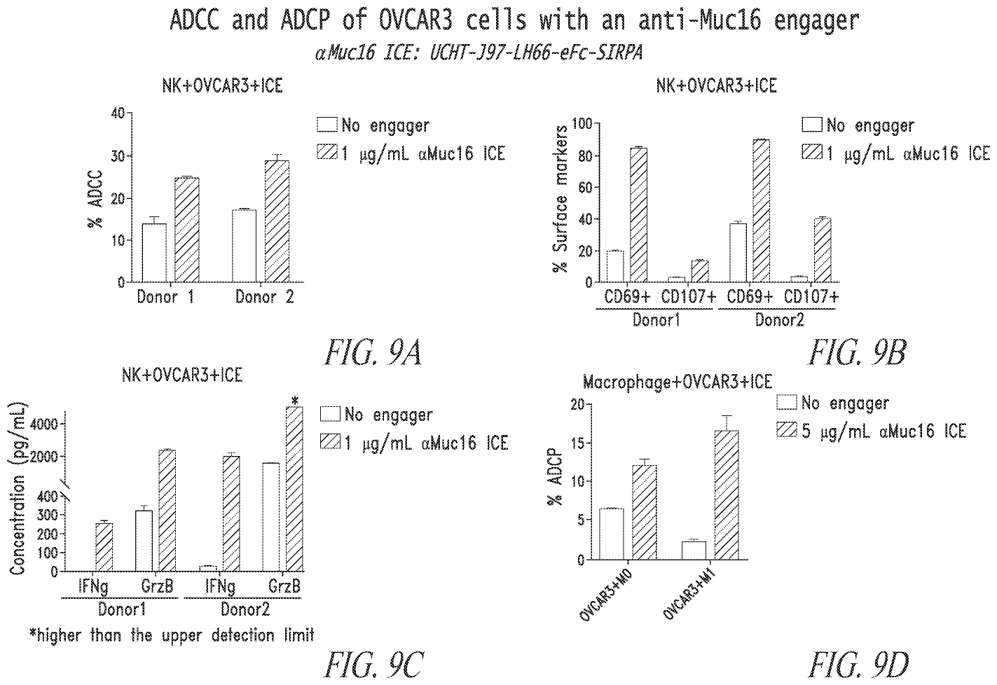

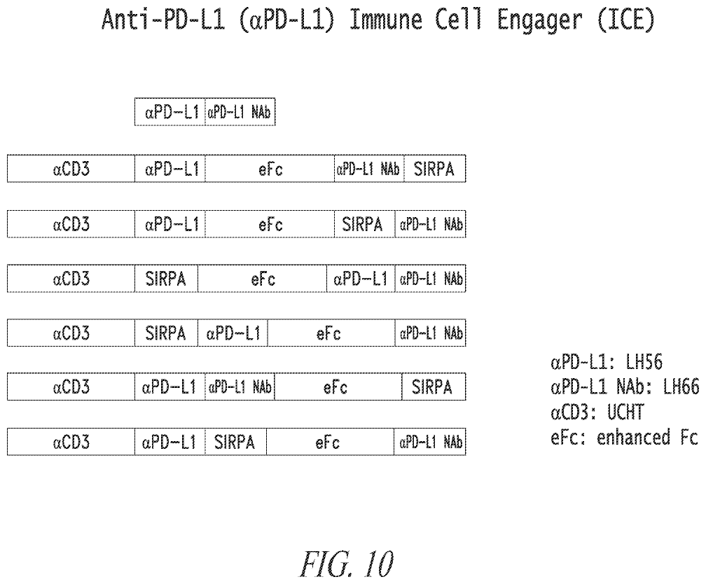

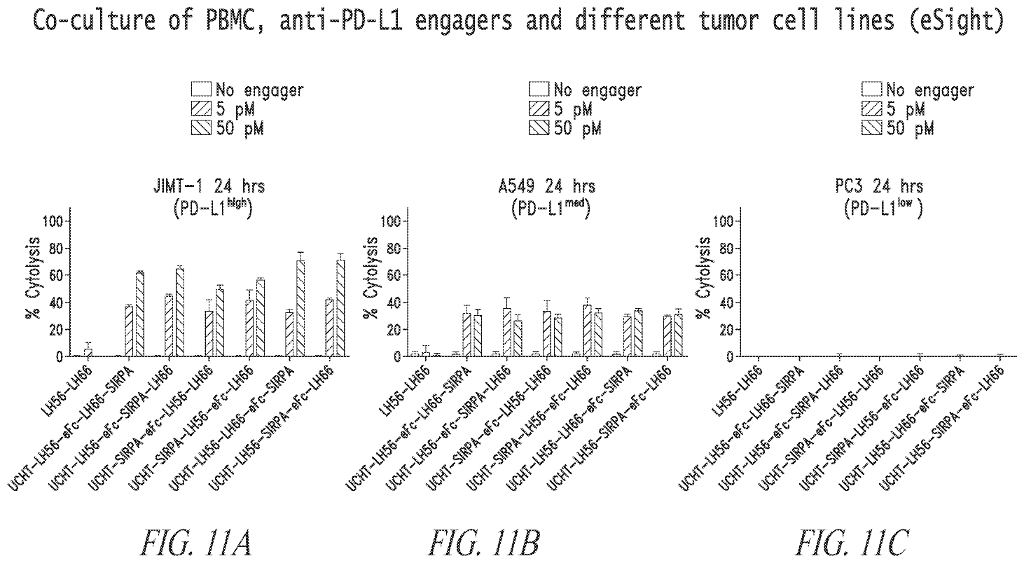

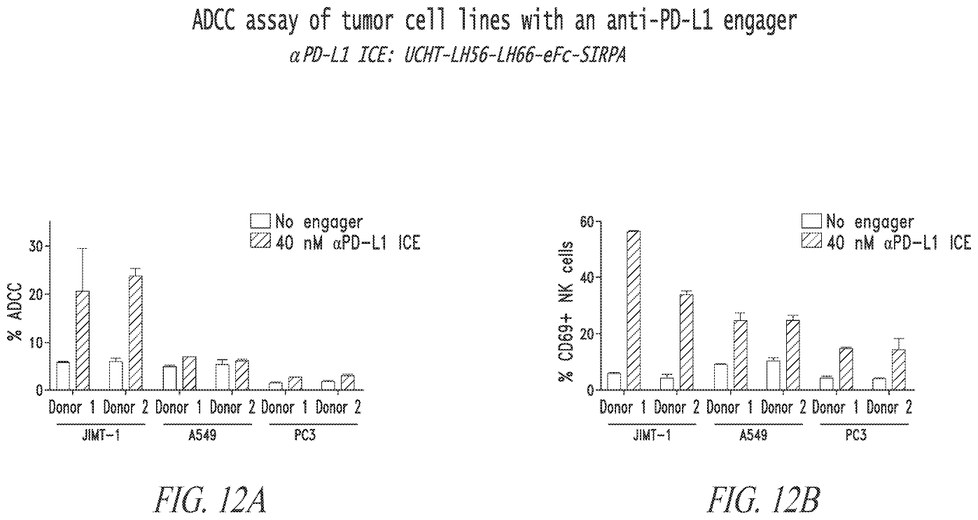

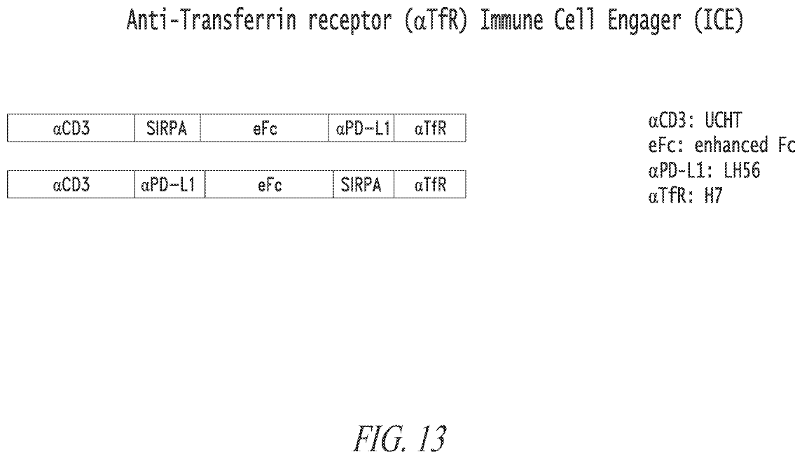

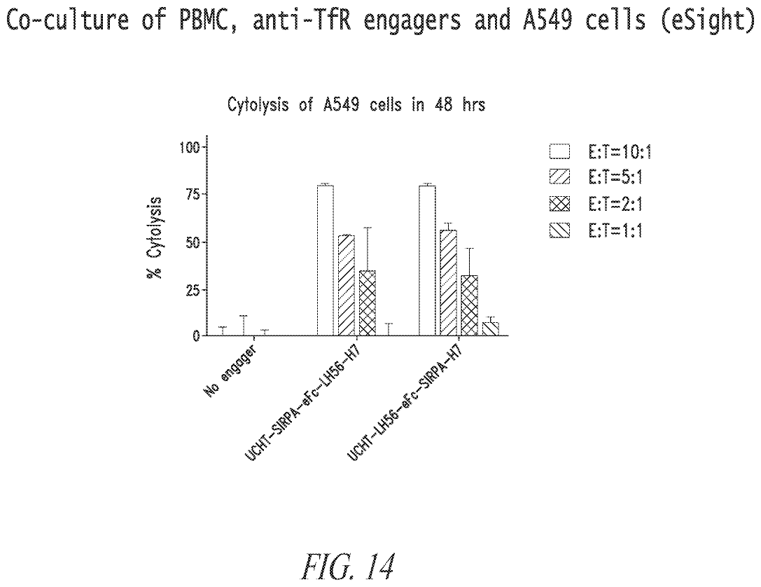

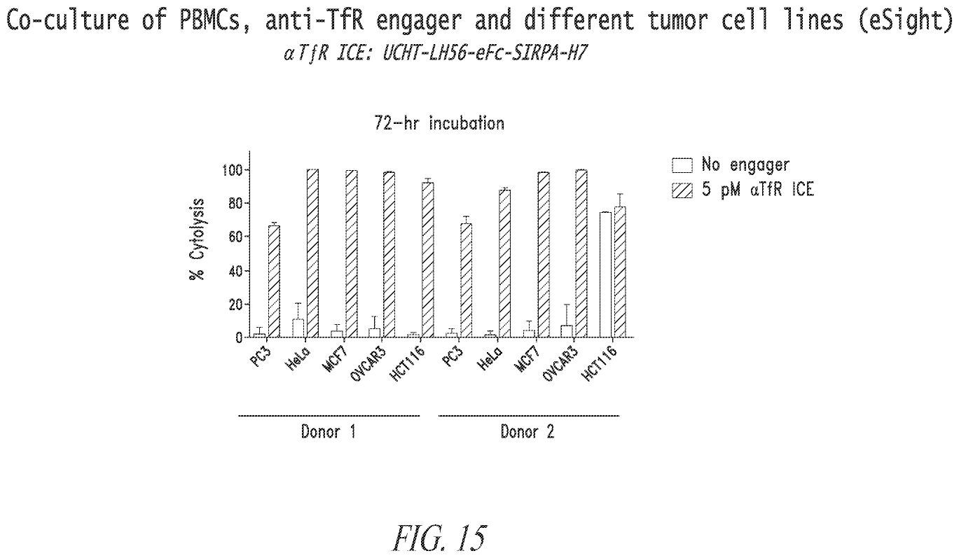

Anti-Muc16 engagers were tested at four different concentrations (no engager, 0.5 pM, 5 pM, or 50 pM) by monitoring cell proliferation with the impedance-based xCELLigence eSight to measure their ability to potentiate cytolysis of tumor cell monolayers including the Muc16 medium expression cell line HeLA (FIGS. 4A, 4B, 5A, 5B) and the Muc16 high expression cell line OVCAR3 (FIGS. 4C, 4D, 5C, 5D) that were co-cultured with PBMC from two different donors. The anti-Muc16 engager UCHT-J97-LH66-eFc-SIRPA was selected for further cytotoxicity testing at a concentration of 5 pM on the Muc16 high expression cell line OVCAR3 (FIG. 6A), the MUC16 medium expression cell line HeLA (FIG. 6B), and the Muc16 low expression cell line PC3 (FIG. 6C) that were co-cultured with PBMC from two different donors and incubated for 24 hours, 48 hours, or 72 hours. Production of the cytokines interferon gamma, IL-2, and granzyme B was measured by ELISA (FIG. 7A), while the presence of the surface activation markers CD69, 4-1BB, OX40, and PD-1 was measured by flow cytometry on CD3+ T cells (FIG. 7B), CD56+ NK cells (FIG. 5C), and CD19+ B cells (FIG. 7D). Cytolysis (FIG. 8A), surface activation markers (FIG. 8B), and cytokine production (FIG. 8C) were also measured after using UCHT-J97-LH66-eFc-SIRPA to treat OVCAR3 cells co-incubated with pan-T cells. UCHT-J97-LH66-eFc-SIRPA was also added to OVCAR3 cells co-incubated with either NK cells or macrophages, followed by quantification of % ADCC (FIG. 9A), NK cell surface activation markers including CD69 and CD107 (FIG. 9B), and cytokine production (FIG. 9C) for the NK-incubated samples, and % ADCP for the engager-treated samples incubated with M0 or M1 macrophages (FIG. 9D).

Anti-PD-L1 engagers were tested at three different concentrations (no engager, 5 pM, or 50 pM) by using eSight assays to measure their ability to potentiate cytolysis of tumor cell monolayers including the PD-L1 high expression cell line JIMT-1 (FIG. 11A), the PD-L1 medium expression cell line A549 (FIG. 11B), and the PD-L1 low expression cell line PC3 (FIG. 11C) that were co-cultured with donor-derived PBMC. The anti-PD-L1 engager UCHT-LH56-LH66-eFc-SIRPA was selected for further testing at two different concentrations (no engager or 40 nM) to determine % ADCC (FIG. 12A) and the proportion of CD69+ NK cells as measured by flow cytometry (FIG. 12B) on the cell lines JIMT-1, A549, and PC3 that were co-cultured with NK cells from two different donors.

Both of the anti-TfR engagers were tested at four different concentrations by using eSight assays to measure their ability to potentiate cytolysis of A549 tumor cell monolayers that were co-cultured with donor-derived PBMC for 48 hours (FIG. 14). The anti-TfR engager UCHT-LH56-eFc-SIRPA-H7 was selected for further testing at two different concentrations (no engager or 5 pM) to quantify effector cell-mediated cytotoxicity following co-incubation of PBMC derived from two different donors with the tumor cell lines PC3, HeLa, MCF7, OVCAR3, and HCT116 (FIG. 15).

Cytolysis was quantified by eSight assays as follows: one day before the experiment, 1.2×10\hat{ }4 tumor cells/well were seeded and incubated in the xCELLigence RTCA analyzer at 37° C., 5% CO₂. On the day of the experiment, donor PBMCs were added to the wells at a 10:1 Effector: Target ratio. Simultaneously, engagers were added to the wells at the specified concentration, ranging from 0.5 pM to 50 pM. Engager-treated cells were incubated at 37° C., 5% CO₂, and cytotoxicity was analyzed after 24 hours, 48 hours, and 72 hours. Flow cytometry and ELISA were performed as follows: one day before the experiment, 1.2×10\hat{ }4 tumor cells/well were seeded and incubated overnight at 37° C., 5% CO₂. On the day of the experiment, donor PBMCs were added to the wells at 10:1 Effector: Target ratio. Simultaneously, engagers were added to the wells at the specified concentration. Engager-treated cells were incubated for 48 hours at 37° C., 5% CO₂ and immune cell surface markers such as CD69, CD107, 4-1BB, OX40, and PD-1 were analyzed using flow cytometry. Cell culture supernatants were used to analyze secretion of interferon gamma (IFNg) and IL-2 using ThermoFisher ELISA kits and Granzyme B (GrzB) production using an R&D Systems ELISA kit according to the manufacturer's instructions. Antibody-dependent cellular cytotoxicity (ADCC) was quantified as follows: one day before the experiment, tumor cells were stained with CellTrace Far Red, seeded at 2.5×10\hat{ }4 cells/well, and incubated overnight at 37° C., 5% CO₂. On the day of the experiment, donor NK cells were added at a 5:1 Effector: Target ratio. Simultaneously, 1 μg/ml of the engager was added to the wells. Engager-treated cells were incubated overnight at 37° C., 5% CO₂ and analyzed by flow cytometry using CD56 and CellTrace staining to discriminate between NK cells and tumor cells, respectively. 7-AAD was used as the cell apoptosis marker for ADCC of the tumor cells. CD69 and CD107a were used to determine the activation of NK cells. The cell culture supernatant was used to analyze secretion of interferon gamma (IFNg) using a ThermoFisher ELISA kit and Granzyme B (GrzB) production using an R&D Systems ELISA kit according to the manufacturer's instructions. Antibody-dependent cellular phagocytosis (ADCP) was quantified as follows: frozen monocytes were thawed and differentiated into macrophages with 50 ng/ml GM-CSF for 6 days. The differentiated monocytes (M0) were either treated with 20 ng/ml IFNg+50 ng/ml LPS for 24 hours to trigger M1 polarization or left untreated. On the day of the experiment, the tumor cells were labeled with CellTrace Far Red and 3×10\hat{ }4 cells/well of tumor cells were incubated with 5 μg/ml of the engager for 15 minutes at room temperature with gentle rocking. After the incubation, macrophages were added to the tumor cells at a 1:1 ratio and incubated for 2.5 hours at 37° C., 5% CO₂. The cells were analyzed by flow cytometry using CD14 and CellTrace staining to discriminate between macrophages and tumor cells, respectively. The phagocytic population was double positive for CD14 and CellTrace Far Red.

Results: All tested immune cell engagers targeting Muc16 produced a dose-dependent increase in cytolysis observed in tumor cells co-incubated with PBMC, while cytolysis of control cells not treated with the engagers was minimal. The best performing anti-Muc16 engager (UCHT-J97-LH66-eFc-SIRPA) was selected for further testing. A dose of 5 pM of UCHT-J97-LH66-eFc-SIRPA was sufficient to kill approximately 40% of the low Muc16 expression tumor cell line PC3 after a 48-hour incubation period, with cell killing at the same timepoint reaching 80% in the medium Muc16 expression tumor cell line HeLa and around 100% in the high Muc16 expression tumor cell line OVCAR3. 5 PM of UCHT-J97-LH66-eFc-SIRPA successfully stimulated PBMCs to produce significant amounts of the proinflammatory cytokines interferon gamma and granzyme B as determined by ELISA, and flow cytometry analysis of the lymphocyte population revealed a dramatic enrichment in surface activation markers on CD3+ T cells and CD56+ NK cells, with a trend towards increased activation of CD19+ B cells. Using 5 PM of UCHT-J97-LH66-eFc-SIRPA to treat tumor cells co-incubated with pan-T cells likewise led to increased cytolysis, cytokine production, and lymphocyte activation as indicated by activation markers such as CD69, 4-1BB, and PD-1. Similar tests were performed with tumor cells co-incubated with NK cells or macrophages and treated with UCHT-J97-LH66-eFc-SIRPA. With NK cells, exposure to only 1 μg/ml of engager was sufficient to almost double the ADCC on OVCAR3 cells compared to controls not exposed to the engager. More than 80% of NK cells were positive for CD69 after engager treatment, while simultaneously producing significantly elevated amounts of interferon gamma and granzyme B. Engager-exposed macrophages also displayed enhanced ADCP of tumor cells, with the ADCP values nearly doubling for undifferentiated M0 macrophages and increasing approximately seven-fold for pro-inflammatory M1 macrophages.

The seven tested anti-PD-L1 engagers displayed increased cytolysis of both the medium PD-L1 expressing tumor cell line A549 and the high PD-L1 expressing cell line JIMT-1 at the 24-hour time point after co-incubation with donor-derived PBMCs. Both low and high doses of engager performed similarly on A549 cells, while a dose-dependent improvement in cytolysis was observed on JIMT-1 cells. No cytolysis was observed at any dose on the low PD-L1 expressing tumor cell line PC3. The best performing anti-PD-L1 engager (UCHT-LH56-LH66-eFc-SIRPA) was selected for further testing. 40 nM of UCHT-LH56-LH66-eFc-SIRPA successfully stimulated NK cells to increase ADCC in JIMT-1 cells compared to untreated controls. While ADCC was less evident in the lower PD-L1 expressing cell lines A549 and PC3, all three tested cell lines exhibited increased NK cell activation as determined by flow cytometric quantification of the proportion of CD69+ NK cells.

Two anti-TfR engagers were tested for their ability to facilitate A549 tumor cell killing after co-incubation with PBMCs. The engager-mediated cell cytolysis was correlated with the E:T ratio for both, with the highest 10:1 E:T ratio enabling destruction of more than 80% of the tumor cell monolayer contrasted with near undetectable cytolysis for the non-engager-treated control. The best performing anti-TfR engager (UCHT-LH56-eFc-SIRPA-H7) was selected for cytolysis testing on a broader panel of five different tumor cell lines, resulting in cell killing rates ranging from approximately 65% to 100% when co-incubated with PBMC.

Conclusions: We evaluated 14 different anti-Muc16 immune cell engagers, 7 different anti-PD-L1 immune cell engagers, and 2 different anti-TfR immune cell engagers by co-incubating them with tumor cells and immune cells and quantifying effector cell-mediated cytolysis, cytokine production, presence of surface activation markers, NK-mediated ADCC, and macrophage-mediated ADCP. One engager was selected from each group for additional testing, displaying strong performance in all tested categories. Overall, the results demonstrate that our multivalent approach to engager design is successful in promoting robust engager binding to target cells and immune cell activation, leading to immune effector cell-mediated cell killing of engager-decorated cells.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein

It is also to be understood that as used herein and in the appended claims, the singular forms “a,” “an,” and “the” include plural reference unless the context clearly dictates otherwise, the term “X and/or Y” means “X” or “Y” or both “X” and “Y,” and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1. A recombinant herpes simplex virus comprising a modified oncolytic herpes virus genome, wherein the modified herpes virus genome comprises an expression cassette encoding an immune cell engager protein, wherein the immune cell engager protein comprises a T-cell binding domain and a tumor-associated antigen binding domain, wherein the T-cell binding domain comprises an anti-CD3, anti-CD28, anti-OX40, or anti-4-1BB antibody or antibody fragment thereof.

2. (canceled)

3. The recombinant herpes simplex virus of claim 1, wherein the anti-CD3 antibody or fragment thereof is selected from the group consisting of UCHT1 (SEQ ID NO: 1), SP34 (SEQ ID NO:2), and CRIS-7 (SEQ ID NO:3).

4. The recombinant herpes simplex virus of claim 1, wherein the tumor-associated antigen binding domain comprises an antibody or fragment thereof, wherein the antibody or fragment thereof comprises a scFv or a nanobody.

5. (canceled)

6. The recombinant herpes simplex virus of claim 4, wherein the antibody or fragment thereof specifically binds to a tumor-associated antigen selected from the group consisting of B7-H3, B7-H6, CD70, CEA, CSPG4, EGFRvIII, EphA2, EpCAM, EGFR, ErbB2 (HER2), FAP, FRα, GD2, GD3, HLA-A1+MAGE1, IL-11Rα, IL-13Rα2, Lewis-Y, Mesothelin, Muc1, Muc16, NKG2D, PSMA, ROR1, TAG72, VEGFR2, TfR1, GLUT1, or PD-L1.

7. (canceled)

8. The recombinant herpes simplex virus of claim 1, wherein the T-cell binding domain and the tumor-associated antigen binding domain are joined by a flexible linker comprising one or more repeats of a peptide comprising the amino acid sequence GGGGS (SEQ ID NO: 4).

9. The recombinant herpes simplex virus of claim 1, wherein the expression cassette comprises an EF1α promoter controlling expression of the immune cell engager protein.

10. The recombinant herpes simplex virus of claim 1, wherein the immune cell engager protein is capable of being secreted from a host cell infected by the recombinant herpes simplex virus.

11. The recombinant herpes simplex virus of claim 1, further comprising at least one miRNA target sequence operably linked to a first copy of an ICP34.5 gene, and a second copy of the ICP34.5 gene comprises an inactivating mutation.

12. (canceled)

13. (canceled)

14. The recombinant herpes simplex virus of claim 11, wherein the miRNA target sequences are inserted into a 3′ untranslated region of the first copy of the ICP34.5 gene.

15. (canceled)

16. The recombinant herpes simplex virus of claim 11, wherein the at least one miRNA target sequence comprises miR-124 (SEQ ID NO:6) and miR-143 (SEQ ID NO:7).

17. The recombinant herpes simplex virus of claim 1, wherein the modified herpes virus genome comprises additional mutations or modifications in viral genes ICP4, ICPO, ICP27, ICP47, and/or LAT.

18. (canceled)

19. The recombinant herpes simplex virus of claim 17, wherein said virus is modified by replacing one or more native viral promotors with a heterologous promoter, and wherein said modification is replacement of the entire promoter-regulatory region of ICP27 with a tumor specific promoter.

20. The recombinant herpes simplex virus of claim 19, wherein the ICP27 promoter is replaced with a CEA promoter (SEQ ID NO:13).

21. (canceled)

22. The recombinant herpes simplex virus of claim 1, further comprising at least one nucleic acid encoding a non-viral protein selected from the group consisting of immunostimulatory factors, antibodies, and checkpoint blocking peptides, wherein the at least one nucleic acid is operably linked to a generic or a tumor-specific promoter.

23. The recombinant herpes simplex virus of claim 22, wherein the non-viral protein is selected from the group consisting of IL12 (SEQ ID NO:9), IL15 (SEQ ID NO:10), and IL15 receptor alpha subunit (SEQ ID NO:11).

24. The recombinant herpes simplex virus of claim 22, wherein the tumor-specific promoter is promoter is CRCX4 (SEQ ID NO:8).

25. The recombinant herpes simplex virus of claim 1, further comprising a nucleic acid sequence encoding a fusogenic form of glycoprotein B.

26. (canceled)

27. The recombinant herpes simplex virus of claim 1, wherein the oncolytic herpes virus is HSV-1.

28. An immune cell engager protein comprising a T-cell binding domain and a tumor-associated antigen binding domain, wherein said tumor-associated antigen binding domain is anti-CEACAM6, anti-Muc16, anti-PD-L1, or anti-TfR1, wherein said T-Cell binding domain is an anti-CD3, anti-CD28, anti-OX40, or an anti-4-1BB antibody or antibody fragment.

29. (canceled)

30. The immune cell engager protein according to claim 28 wherein said anti-CD3 antibody or fragment thereof is selected from the group consisting of UCHT1 (SEQ ID NO:1), SP34 (SEQ ID NO:2), and CRIS-7 (SEQ ID NO:3).

31. The immune cell engager protein according to claim 28 wherein said T-cell binding domain and the tumor-associated antigen binding domain are joined by a flexible linker, wherein said flexible linker comprises one or more repeats of a peptide comprising the amino acid sequence GGGGS (SEQ ID NO:4).

32. (canceled)

33. An isolated nucleic acid molecule encoding an immune cell engager protein according to claim 28.

34. An expression vector which directs the expression of a nucleic acid sequence according to claim 33.

35. (canceled)

36. A pharmaceutical composition comprising an immune cell engager protein according to claim 28 and a pharmaceutically acceptable carrier.

37. A method for treating cancer in a subject suffering therefrom, comprising the step of administering to a subject a therapeutically effective amount of the pharmaceutical composition according to claim 36.

38. (canceled)


SEQUENCE LISTING (13 sequences)

SEQ ID NO: 1 - protein, 249 aa, Homo sapiens
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 2 - protein, 250 aa, Homo sapiens
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGSGGGSGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 3 - protein, 249 aa, Homo sapiens
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAGGGGSGGGGSGGGGSGGGGSAQQVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 4 - protein, 5 aa, synthetic construct
GGGGS

SEQ ID NO: 5 - DNA, 538 bp, Herpes simplex virus
  source 1..538: mol_type = genomic DNA
cgtcacatccaggccggcggaaaccggaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcatatccacccccgcccgggtccccgcggaagcggaacggtgtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtc

SEQ ID NO: 6 - DNA, 20 bp, Homo sapiens
  source 1..20: mol_type = genomic DNA
ggcattcaccgcgtgcctta

SEQ ID NO: 7 - DNA, 22 bp, Homo sapiens
  source 1..22: mol_type = genomic DNA
tgagctacagtgcttcatctca

SEQ ID NO: 8 - DNA, 279 bp, Homo sapiens
  source 1..279: mol_type = genomic DNA
ttaccgaccacccgcaaacagcagggtcccctgggcttcccaagccgcgcacctctccgccccgcccctgcgccctccttcctcgcgtctgcccctctcccccaccccgccttctccctccccgccccagcggcgcatgcgccgcgctcggagcgtgtttttataaaagtccggccgcggccagaaacttcagtttgttggctgcggcagcaggtagcaaagtgacgccgagggcctgagtgctccagtagccaccgcatctggagaaccagcggttac

SEQ ID NO: 9 - protein, 554 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 12 - protein, 19 aa, synthetic construct
ATNFSLLKQAGDVEENPGP

SEQ ID NO: 13 - DNA, 432 bp, Homo sapiens
  source 1..432: mol_type = genomic DNA
tagtgccctggagagcatggggagacccgggaccctgctgggtttctctgtcacaaaggaaaataatccccctggtgtgacagacccaaggacagaacacagcagaggtcagcactggggaagacaggttgtcctcccaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactcaagctcttctccacagaggaggacagagcagacagcagagacc